(19) United States
(12) Patent Application Publication
(10) Pub. No.: US 2010/0098693 A1
(43) Pub. Date: Apr. 22, 2010

(54) COMPOSITIONS AND METHODS FOR BLOOD-BRAIN BARRIER DELIVERY OF ORGANOPHOSPHATASES

(76) Inventors: William M. Pardridge, Pacific Palisades, CA (US); Ruben J. Boado, Agoura Hills, CA (US)

Correspondence Address: WILSON, SONSINI, GOODRICH & ROSATI, 650 PAGE MILL ROAD, PALO ALTO, CA, 94304-1050, US

(21) Appl. No.: 12/574,571

(22) Filed: Oct. 6, 2009

Related U.S. Application Data
(60) Provisional application No. 61/103,510, filed on Oct. 7, 2008.

(51) Int. Cl. A61K 39/395; C07K 16/18; A61P 25/28

(52) U.S. Cl. 424/133.1; 530/387.3

(57) ABSTRACT

Provided herein are compositions and related methods for delivering an organophosphatase to the CNS. The methods include systemic administration of a bifunctional fusion antibody comprising an antibody to a receptor expressed on the surface of the blood-brain barrier (BBB receptor) and an organophosphatase. In some embodiments, the compositions described herein are used to treat a subject suffering from or at high risk of exposure to an organophosphate (e.g., a nerve gas).

### CROSS-REFERENCE

This application claims the benefit of U.S. Provisional Application No. 61/103,510, filed Oct. 7, 2008, which application is incorporated herein by reference.

### STATEMENT AS TO FEDERALLY SPONSORED RESEARCH

This invention was made with the support of the United States government under Grant number U44-NS-57843 by the National Institutes of Health. The United States Government may have certain rights in the invention.

### BACKGROUND OF THE INVENTION

Paraoxonase (PON)-1 is the most potent human organophosphatase for acute treatment of intoxication with organophosphates, including chemical nerve gas agents, which attack the nervous system, as well as for the chronic treatment of cerebral atherosclerosis. However, PON1, like other large molecule drugs, does not cross the blood-brain barrier (BBB) to enter the central nervous system (CNS) when administered systemically. Thus, what is needed is a way to deliver PON1 to the CNS where it can act most effectively.

### SUMMARY OF THE INVENTION

Described herein are compositions and related methods for delivering PON1 across the BBB to the CNS in a subject in need thereof. In particular, the methods allow delivery of PON1 to the CNS by systemically administering a therapeutically effective amount of a bifunctional fusion antibody that comprises an antibody that binds to the extracellular domain of a receptor expressed on the surface of BBB and an organophosphatase enzyme.

Accordingly, in one aspect provided herein is a method for treating organophosphate intoxication (e.g., acute organophosphate intoxication or chronic organophosphate intoxication) in a subject in need thereof, comprising systemically administering to the subject a dose of a fusion antibody having organophosphatase activity and binding to the extracellular domain of a receptor expressed on the BBB. In some embodiments, the receptor expressed on the BBB is an insulin receptor, a transferrin receptor, or a lipoprotein receptor. In some embodiments at least about 0.5% of the systemically administered dose is delivered to the brain. In some embodiments, the fusion antibody used in this method comprises an amino acid sequence at least 70% identical to the amino acid sequence of human PON1. In some embodiments, the fusion antibody used in this method comprises an immunoglobulin heavy chain comprising a CDR1 corresponding to the amino acid 45-54 of SEQ ID NO:21 with up to 4 single amino acid mutations, a CDR2 corresponding to the amino acid s 69-85 of SEQ ID NO:21 with up to 6 single amino acid mutations, or a CDR3 corresponding to the amino acid s 118-121 of SEQ ID NO:21 with up to 3 single amino acid mutations, wherein the single amino acid mutations are substitutions, deletions, or insertions.

In another aspect provided herein is a method for protecting a subject from organophosphate intoxication comprising systemically administering to a subject at high risk of organophosphate intoxication a dose of a fusion antibody having organophosphatase activity and binding to the extracellular domain of a receptor expressed on the BBB. In some embodiments, the receptor expressed on the BBB is an insulin receptor, a transferrin receptor, or a lipoprotein receptor. In some embodiments, the fusion antibody used in this method comprises an amino acid sequence at least 70% identical to the amino acid sequence of human PON1. In some embodiments at least about 0.5% of the systemically administered dose is delivered to the brain.

In another aspect provided herein is a method for treating of cerebral atherosclerosis, comprising administering to a subject in need thereof a therapeutically effective dose of a fusion antibody having organophosphatase activity and binding to the extracellular domain of a receptor expressed on the BBB.

In yet another aspect provided herein is a fusion antibody comprising a heavy chain immunoglobulin or a light chain immunoglobulin covalently linked to an organophosphatase, wherein the fusion antibody binds to the extracellular domain of a receptor expressed on the BBB. In some embodiments, the receptor expressed on the BBB is an insulin receptor, a transferrin receptor, or a lipoprotein receptor. In some embodiments, the fusion antibody used in this method comprises an amino acid sequence at least 70% identical to the amino acid sequence of human PON1. In some embodiments, the fusion antibody used in this method comprises an immunoglobulin heavy chain comprising CDR1 corresponding to the amino acids 45-54 of SEQ ID NO:21 with up to 4 single amino acid mutations, a CDR2 corresponding to the amino acids 69-85 of SEQ ID NO:21 with up to 6 single amino acid mutations, or a CDR3 corresponding to the amino acids 118-121 of SEQ ID NO:21 with up to 3 single amino acid mutations, wherein the single amino acid mutations are substitutions, deletions, or insertions. In some embodiments, the fusion antibody competes for binding to the human insulin receptor with a fusion antibody comprising the amino acid sequence of SEQ ID NO:23.

In a further aspect, provided herein is a nucleic acid comprising (i) a first sequence encoding a heavy chain immunoglobulin and an organophosphatase in frame with the heavy chain immunoglobulin; (ii) a second sequence encoding a light chain immunoglobulin and an organophosphatase in frame with the light chain immunoglobulin; or (iii) the complementary sequence of (i) or (ii), where the heavy chain and light chain immunoglobulin are from an antibody against a BBB receptor (e.g., a human insulin receptor, transferrin receptor, or lipoprotein receptor). In some embodiments, the nucleic acid comprises (i) and a nucleic acid sequence encoding an immunoglobulin light chain or its complementary sequence. In some embodiments, the nucleic acid comprises (ii) and a nucleic acid sequence encoding an immunoglobulin heavy chain or its complementary sequence. In some embodiments, the amino acid sequence of the encoded organophosphatase comprises an amino acid sequence at least 70% identical to the amino acid sequence corresponding to SEQ ID NO:19. In some embodiments, the nucleic acid hybridizes, under high stringency conditions, to a nucleic acid sequence encoding the amino acid sequence of SEQ ID NO:23. In some embodiments provided herein are vectors comprising any of the foregoing nucleic acids. In some embodiments provided herein is a cell (e.g., a mammalian cell) comprising any of the foregoing nucleic acids or vectors.

In a further aspect provided herein is a method of manufacturing a fusion antibody, comprising integrating into a eukaryotic cell (e.g., a mammalian cell) a single tandem expression vector comprising a sequence encoding a heavy chain immunoglobulin and a sequence encoding a light chain immunoglobulin, wherein either the sequence encoding the heavy chain immunoglobulin, or the sequence encoding the light chain immunoglobulin further comprises a sequence encoding an organophosphatase fused in frame with the encoded heavy chain immunoglobulin or the encoded light chain immunoglobulin, where the heavy chain and light chain immunoglobulin are from an antibody against a BBB receptor (e.g., a human insulin receptor, transferrin receptor, or lipoprotein receptor). In some embodiments, the nucleic acid encodes the heavy chain immunoglobulin fused in frame with the organophosphatase. In some embodiments, the nucleic acid encodes the light chain immunoglobulin fused in frame with the organophosphatase.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings, as follow:

FIG. 1. The HIRMAb-PON1 fusion protein is formed by fusion of the amino terminus of human PON1 to the carboxyl terminus of the heavy chain of the HIRMAb. The fusion protein is a bi-functional molecule: the fusion protein binds the HIR, at the BBB, to mediate transport into the brain, and has PON1 organophosphatase enzyme activity.

FIG. 2. (A) Ethidium bromide stain of agarose gel of human PON1 cDNA (lane 1), which was produced by PCR from cDNA produced by reverse transcription of RNA from human liver, and PON1-specific ODN primers (Table I). The expected band of ˜1.1 kb band corresponding to the human PON1 cDNA is shown in lane 1. DNA standards are PhiX174 HaeIII digested DNA, 1.4, 1.0, 0.8, 0.6, 0.3-0.1 kb (lane 2); and Lambda HindIII digested DNA, 23, 9.4, 6.6, 4.4, 2.3, 2.0 and 0.56 kb (lane 3). (B) Genetic engineering of pHIRMAb-PON1, the eukaryotic expression plasmid encoding the fusion protein of PON1 and the heavy chain (HC) of the chimeric HIRMAb. The fusion gene is 5′-flanked by the cytomegalovirus (CMV) promoter and 3′-flanked by the bovine growth hormone polyA (pA) sequence.

FIG. 3. Western blot with either anti-human IgG primary antibody (A) or an anti-human PON1 primary antiserum (B). The immunoreactivity of the HIRMAb-PON1 fusion protein (lane 2) is compared to the chimeric HIRMAb (lane 3) and to recombinant PON1 (lane 1). Both the HIRMAb-PON1 fusion protein and the HIRMAb have identical light chains on the anti-hIgG Western. The HIRMAb-PON1 fusion heavy chain reacts with both the anti-IgG and the anti-human PON1 antibody, whereas the HIRMAb heavy chain only reacts with the anti-IgG antibody. The size of the HIRMAb-PON1 fusion heavy chain, 95 kDa, is about 40 kDa larger than the size of the heavy chain of the HIRMAb, owing to the fusion of the 40 kDa PON1 to the 55 kDa HIRMAb heavy chain.

FIG. 4. Binding of either the chimeric HIRMAb or the HIRMAb-PON1 fusion protein to the HIR extracellular domain (ECD) is saturable. The ED50 of HIRMAb-PON1 binding to the HIR ECD is comparable to the ED50 of the binding of the chimeric HIRMAb.

FIG. 5. Genetic engineering of tandem vector (TV) encoding the intact, hetero-tetrameric HIRMAb-PON1 fusion protein, designated HIRMAb-PON1 TV. The mature PON1 cDNA, produced by PCR, is inserted in the HpaI site at the 3′-end of the HC of the HIRMAb, expressed by the universal TV or UTV. The TV contains 3 separate expression cassettes encoding for the HIRMAb light chain (LC), the fusion gene of the HIRMAb heavy chain (HC) and PON1, and for dihydrofolate reductase (DHFR).

FIG. 6. Domain structure of heavy chain of the HIRMAb-PON1 fusion protein. The heavy chain of the HIRMAb is comprised of 3 complementarity determining regions (CDRs) and 4 framework regions (FRs), which form the variable region of the heavy chain (VH). The VH is fused to the constant region of human IgG1, which is comprised of the CH1 region, the hinge region, the CH2 region, followed by the CH3 region, which is fused, via a Ser-Ser linker, to the amino terminus of human paraoxonase (PON1)-1. The single N-linked glycosylation site within CH2 and the 4 N-linked glycosylation sites within PON1 are underlined. The Met-55 and the Arg-192 polymorphisms of PON1 are bold/underlined.

FIG. 7. Domain structure of light chain of the HIRMAb-PON1 fusion protein. The light chain of the HIRMAb is comprised of 3 complementarity determining regions (CDRs) and 4 framework regions (FRs), which form the variable region of the light chain (VL). The VL is fused to the constant region of human IgG kappa.

FIG. 8. Trojan horse (TH) accesses receptor (R1) to deliver enzyme (E) across both the BBB and the brain cell membrane. The enzyme converts substrate (S) to product (P) inside brain cells.

### DETAILED DESCRIPTION OF THE INVENTION

I. Introduction

II. Some Definitions

III. The Blood Brain Barrier

IV. Organophosphatase Fusion Antibodies for transport across the BBB

V. Compositions

VI. Nucleic acids, vectors, cells, and manufacture

VII. Methods

### ABBREVIATIONS

AA amino acid
ACE acetylcholinesterase
AP alkaline phosphatase
BBB blood-brain barrier
BCA bicinchoninic acid
BGH bovine growth hormone
CDR complementarity determining region
CHO Chinese hamster ovary
CMV cytomegalovirus
DC dilutional cloning
DEPFMU diethylphospho-6,8-difluoro-4-methyl-umbelliferone
DHFR dihydrofolate reductase
E enzyme
ECD extracellular domain
ED50 effective dose causing 50% saturation
FR framework region
FS flanking sequence
FWD forward
HC heavy chain
HC-PON1 PON1 fused to carboxyl terminus of HIRMAb HC
HIR human insulin receptor
HIRMAb MAb to HIR
HIRMAb HC heavy chain of HIRMAb
HIRMAb LC light chain of HIRMAb
HIRMAb-PON1 fusion protein of HIRMAb and PON1, where PON1 is fused to the HC carboxyl terminus
HPLC high pressure liquid chromatography
HT hypoxanthine-thymidine
ID injected dose
IgG immunoglobulin G
LC light chain
MAb monoclonal antibody
MAH mouse anti-human IgG
MTX methotrexate
MW molecular weight
N asparagine
nt nucleotide
ODN oligodeoxynucleotide
OP organophosphate
orf open reading frame
pA poly-adenylation
PAGE polyacrylamide gel electrophoresis
PBS phosphate buffered saline
PBST PBS plus Tween-20
PCR polymerase chain reaction
PON1 paraoxonase-1
PON1-HC PON1 fused to amino terminus of HIRMAb HC
PON1-HIRMAb fusion protein of HIRMAb and PON1, where PON1 is fused to the HC amino terminus
pI isoelectric point
R receptor
REV reverse
RNase A ribonuclease A
RT reverse transcriptase
RT room temperature
SDM site-directed mutagenesis
SDS sodium dodecyl sulfate
SEC size exclusion chromatography
Ser serine
SFM serum free medium
TH Trojan horse
TV tandem vector
UTV universal TV
VH variable region of heavy chain
VL variable region of light chain

### I. INTRODUCTION

The blood brain barrier is a severe impediment to the delivery of systemically administered organophosphatases to the central nervous system, the primary target of organophosphate nerve gas agents. The compositions and methods described herein address three factors that are important in delivering a therapeutically significant level of organophosphatase activity across the BBB to the CNS: 1) modification of an organophosphatase to allow it to cross the BBB; 2) the amount and rate of uptake of systemically administered modified organophosphatase into the CNS, and 3) retention of organophosphatase activity once across the BBB. Various aspects of the methods and compositions described herein address these factors, by providing fusion antibodies, that can be administered systemically, comprising an organophosphatase (i.e., a protein having organophosphatase activity) fused, with or without an intervening linker sequence, to an immunoglobulin (heavy chain or light chain) directed against the extracellular domain of a receptor (e.g., a human insulin receptor) expressed on the BBB.

Accordingly, the invention provides compositions and methods for delivering an exogenous organophosphatase to the central nervous system of a subject in need, e.g., a subject suffering from or at high risk of nerve gas intoxication, by systemically administering to a subject in need thereof a therapeutically effective dose of a bifunctional fusion antibody comprising an antibody against a receptor expressed on the BBB (e.g., an hIR) and an organophosphatase (e.g., human PON1).

### II. SOME DEFINITIONS

The term “antibody” describes an immunoglobulin whether natural or partly or wholly synthetically produced. The term also covers any polypeptide or protein having a binding domain which is, or is homologous to, an antigen-binding domain. CDR grafted antibodies are also contemplated by this term.

“Native antibodies” and “native immunoglobulins” are usually heterotetrameric glycoproteins of about 150,000 daltons, composed of two identical light (L) chains and two identical heavy (H) chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“VH”) followed by a number of constant domains (“CH”). Each light chain has a variable domain at one end (“VL”) and a constant domain (“CL”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

The term “variable domain” refers to protein domains that differ extensively in sequence among family members (i.e. among different isoforms, or in different species). With respect to antibodies, the term “variable domain” refers to the variable domains of antibodies that are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the “framework region” or “FR”. The variable domains of unmodified heavy and light chains each comprise four FRs (FR1, FR2, FR3 and FR4, respectively), largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., (1991), Sequences of Proteins of Immunological Interest, 5th Ed. Public Health Service, National Institutes of Health, Bethesda, Md., pages 647-669). The constant domains are not involved directly in binding an antibody to an antigen, but exhibit various effector functions, such as participation of the antibody in antibody-dependent cellular toxicity.

The term “hypervariable region” when used herein refers to the amino acid residues of an antibody which are responsible for antigen-binding. The hypervariable region comprises amino acid residues from three “complementarily determining regions” or “CDRs”, which directly bind, in a complementary manner, to an antigen and are known as CDR1, CDR2, and CDR3 respectively. In the light chain variable domain, the CDRs typically correspond to approximately residues 24-34 (CDRL1), 50-56 (CDRL2) and 89-97 (CDRL3), and in the heavy chain variable domain the CDRs typically correspond to approximately residues 31-35 (CDRH1), 50-65 (CDRH2) and 95-102 (CDRH3); Kabat et al., (1991), Sequences of Proteins of Immunological Interest, 5th Ed. Public Health Service, National Institutes of Health, Bethesda, Md.) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk, (1987), J. Mol. Biol., 196:901-917.

As used herein, “variable framework region” or “VFR” refers to framework residues that form a part of the antigen binding pocket or groove and/or that may contact antigen. In some embodiments, the framework residues form a loop that is a part of the antigen binding pocket or groove. The amino acids residues in the loop may or may not contact the antigen. In an embodiment, the loop amino acids of a VFR are determined by inspection of the three-dimensional structure of an antibody, antibody heavy chain, or antibody light chain. The three-dimensional structure can be analyzed for solvent accessible amino acid positions as such positions are likely to form a loop and/or provide antigen contact in an antibody variable domain. Some of the solvent accessible positions can tolerate amino acid sequence diversity and others (e.g. structural positions) can be less diversified. The three dimensional structure of the antibody variable domain can be derived from a crystal structure or protein modeling. In some embodiments, the VFR comprises, consist essentially of, or consists of amino acid positions corresponding to amino acid positions 71 to 78 of the heavy chain variable domain, the positions defined according to Kabat et al., 1991. In some embodiments, VFR forms a portion of Framework Region 3 located between CDRH2 and CDRH3. The VFR can form a loop that is well positioned to make contact with a target antigen or form a part of the antigen binding pocket.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA, and IgA2. The heavy-chain constant domains (Fc) that correspond to the different classes of immunoglobulins are called α, δ, ε, γ, and μ, respectively. The subunit structures and three-dimensional configurations of different classes of immunoglobulins are well known.

The “light chains” of antibodies (immunoglobulins) from any vertebrate species can be assigned to one of two clearly distinct types, called kappa or (“κ”) and lambda or (“λ”), based on the amino acid sequences of their constant domains.

In referring to an antibody or fusion antibody described herein, the terms “selectively bind,” “selectively binding,” “specifically binds,” or “specifically binding” refer to binding to the antibody or fusion antibody to its target antigen for which the dissociation constant (Kd) is about 10⁻⁶ M or lower, i.e., 10⁻⁷, 10⁻⁸, 10⁻⁹, 10⁻¹⁰, 10⁻¹¹ M, or 10⁻¹²M.

The term antibody as used herein will also be understood to mean one or more fragments of an antibody that retain the ability to specifically bind to an antigen, (see generally, Holliger et al., (2005), Nature Biotech., 23(9):1126-1129). Non-limiting examples of such antibodies include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature, 341:544 546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science, 242:423 426; and Huston et al., (1988) Proc. Natl. Acad. Sci. USA, 85:5879 5883; and Osbourn et al., (1998), Nat. Biotechnol., 16:778). Such single chain antibodies are also intended to be encompassed within the term antibody. Any VH and VL sequences of specific scFv can be linked to human immunoglobulin constant region cDNA or genomic sequences, in order to generate expression vectors encoding complete IgG molecules or other isotypes. VH and VL can also be used in the generation of Fab, Fv or other fragments of immunoglobulins using either protein chemistry or recombinant DNA technology. Other forms of single chain antibodies, such as diabodies are also encompassed.

“F(ab′)2” and “Fab′” moieties can be produced by treating immunoglobulin (monoclonal antibody) with a protease such as pepsin and papain, and includes an antibody fragment generated by digesting immunoglobulin near the disulfide bonds existing between the hinge regions in each of the two H chains. For example, papain cleaves IgG upstream of the disulfide bonds existing between the hinge regions in each of the two H chains to generate two homologous antibody fragments in which an L chain composed of VL (L chain variable region) and CL (L chain constant region), and an H chain fragment composed of VH (H chain variable region) and CHγ1 (γ1 region in the constant region of H chain) are connected at their C terminal regions through a disulfide bond. Each of these two homologous antibody fragments is called Fab′. Pepsin also cleaves IgG downstream of the disulfide bonds existing between the hinge regions in each of the two H chains to generate an antibody fragment slightly larger than the fragment in which the two above-mentioned Fab′ are connected at the hinge region. This antibody fragment is called F(ab′)2.

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

“Single-chain Fv” or “sFv” antibody fragments comprise a VH, a VL, or both a VH and VL domain of an antibody, wherein both domains are present in a single polypeptide chain. In some embodiments, the Fv polypeptide further comprises a polypeptide linker between the VH and VL domains which enables the sFv to form the desired structure for antigen binding. For a review of sFv see, e.g., Pluckthun in The Pharmacology of Monoclonal Antibodies, Vol. 113, Rosenburg and Moore eds. Springer-Verlag, New York, pp. 269 315 (1994).

A “chimeric” antibody includes an antibody derived from a combination of different mammalian sources. The mammal may be, for example, a rabbit, a mouse, a rat, a goat, or a human. The combination of different mammals includes combinations of fragments from human and mouse sources.

In some embodiments, an antibody of the present invention is a monoclonal antibody (MAb), typically a chimeric human-mouse antibody derived by humanization of a mouse monoclonal antibody. Such antibodies are obtained from, e.g., transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesis human antibodies specific for human antigens, and the mice can be used to produce human antibody-secreting hybridomas.

“Treatment” or “treating” as used herein includes achieving a therapeutic benefit and/or a prophylactic benefit. By therapeutic benefit is meant eradication or amelioration of the underlying disorder or condition being treated. For example, in an individual suffering from nerve gas intoxication, therapeutic benefit includes partial or complete halting of symptoms associated with nerve gas neurotoxicity. A prophylactic benefit of treatment includes reducing the risk of a condition, retarding the progress of a condition (e.g., slowing the progression of the effects of an organophosphate neurotoxin), or decreasing the likelihood of occurrence of a condition, e.g., organophosphate-induced paralysis. As used herein, “treating” or “treatment” includes prophylaxis.

As used herein, the term “effective amount” can be an amount, which when administered systemically, is sufficient to effect beneficial or desired results in the CNS, such as neuroprotection against organophosphate neurotoxins, or vascular atherosclerosis. An effective amount is also an amount that produces a prophylactic effect, e.g., an amount that delays, reduces, or eliminates the appearance of an acute pathological or undesired condition. Such conditions include, but are not limited to, acute organophosphate intoxication, chronic organophosphate intoxication, and atherosclerosis. An effective amount can be administered in one or more administrations. In terms of treatment, an “effective amount” of a composition of the invention is an amount that is sufficient to palliate, ameliorate, stabilize, reverse or slow the progression of a disorder, e.g., a neurological disorder. An “effective amount” may be of any of the compositions of the invention used alone or in conjunction with one or more agents used to treat a disease or disorder. An “effective amount” of a therapeutic agent within the meaning of the present invention will be determined by a patient's attending physician or veterinarian. Such amounts are readily ascertained by one of ordinary skill in the art and will a therapeutic effect when administered in accordance with the present invention. Factors which influence what a therapeutically effective amount include, but are not limited to, the organophosphatase specific activity of the organophosphatase fusion antibody administered, its absorption profile (e.g., its rate of uptake into the brain), time elapsed since the initiation of the disorder, and the age, physical condition, metabolism, existence of other disease states, and nutritional status of the individual being treated. Additionally, other medication the patient may be receiving will affect the determination of the therapeutically effective amount of the therapeutic agent to administer.

The term “molecular trojan horse,” as used herein, refers to a molecule that is transported across the BBB, and is capable of acting as a ferry for trans-BBB transport into the CNS when linked covalently or non-covalently to another molecule that does not cross the BBB on its own. Examples of a molecular trojan horse include, but are not limited to, polypeptides (e.g., antibodies) that bind to the ECD of receptors expressed on the BBB, e.g., insulin receptors, transferrin receptors, lipoprotein receptors, or leptin receptors.

A “subject” or an “individual,” as used herein, is an animal, for example, a mammal. In some embodiments a “subject” or an “individual” is a human. In some non-limiting embodiments, the subject suffers acute exposure to organophosphates (e.g., nerve gas), chronic exposure to organophosphates, or vascular atherosclerosis.

In some embodiments, a pharmacological composition comprising an organophosphatase fusion antibody is “administered peripherally” or “peripherally administered.” As used herein, these terms refer to any form of administration of an agent, e.g., a therapeutic agent, to an individual that is not direct administration to the CNS, i.e., that brings the agent in contact with the non-brain side of the blood-brain barrier. “Peripheral administration,” as used herein, includes intravenous, intra-arterial, subcutaneous, intramuscular, intraperitoneal, transdermal, by inhalation, transbuccal, intranasal, rectal, oral, parenteral, sublingual, or trans-nasal.

A “pharmaceutically acceptable carrier” or “pharmaceutically acceptable excipient” herein refers to any carrier that does not itself induce the production of antibodies harmful to the individual receiving the composition. Such carriers are well known to those of ordinary skill in the art. A thorough discussion of pharmaceutically acceptable carriers/excipients can be found in Remington's Pharmaceutical Sciences, Gennaro, A R, ed., 20th edition, 2000: Williams and Wilkins P A, USA. Exemplary pharmaceutically acceptable carriers can include salts, for example, mineral acid salts such as hydrochlorides, hydrobromides, phosphates, sulfates, and the like; and the salts of organic acids such as acetates, propionates, malonates, benzoates, and the like. For example, compositions of the invention may be provided in liquid form, and formulated in saline based aqueous solution of varying pH (5-8), with or without detergents such polysorbate-80 at 0.01-1%, or carbohydrate additives, such mannitol, sorbitol, or trehalose. Commonly used buffers include histidine, acetate, phosphate, or citrate.

A “recombinant host cell” or “host cell” refers to a cell that includes an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome.

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-naturally occurring amino acid, e.g., an amino acid analog. As used herein, the terms encompass amino acid chains of any length, including full length proteins (i.e., antigens), wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refers to compounds that have the same basic chemical structure as a naturally occurring amino acid, i.e., an α carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., (1991), Nucleic Acid Res., 19:5081; Ohtsuka et al., (1985), J. Biol. Chem., 260:2605-2608; and Rossolini et al., (1994), Mol. Cell. Probes 8:91-98).

The terms “isolated” and “purified” refer to a material that is substantially or essentially removed from or concentrated in its natural environment. For example, an isolated nucleic acid may be one that is separated from the nucleic acids that normally flank it or other nucleic acids or components (proteins, lipids, etc. . . . ) in a sample. In another example, a polypeptide is purified if it is substantially removed from or concentrated in its natural environment. Methods for purification and isolation of nucleic acids and proteins are well known in the art.

The term “organophosphatase,” refers to any enzyme (e.g., human PON1), whether naturally occurring or experimentally modified, that hydrolyzes an organophosphate. One unit of organophosphatase activity, as used herein, refers to the hydrolysis of one nmole of organophosphate substrate per hour.

The term “BBB receptor Ab,” refers to an antibody against the extracellular domain of a receptor expressed on the blood-brain barrier. Non-limiting examples of BBB receptors include insulin receptor (e.g. human insulin receptor), transferrin receptor, lipoprotein receptor, and leptin receptor.

### III. THE BLOOD BRAIN BARRIER

As used herein, the “blood-brain barrier” refers to the barrier between the peripheral circulation and the brain and spinal cord which is formed by tight junctions within the brain capillary endothelial plasma membranes, creates an extremely tight barrier that restricts the transport of molecules into the brain, even molecules as small as urea, molecular weight of 60 Da. The blood-brain barrier within the brain, the blood-spinal cord barrier within the spinal cord, and the blood-retinal barrier within the retina, are contiguous capillary barriers within the central nervous system (CNS), and are collectively referred to as the blood-brain barrier or BBB. The BBB severely limits the therapeutic efficacy of an exogenous organophosphatase when administered alone. The methods described herein permit a functional organophosphatase to cross the BBB from the peripheral blood into the CNS following systemic administration of an organophosphatase fusion antibody composition described herein. The methods described herein exploit the expression receptors expressed on the surface of the BBB (e.g., human insulin receptors) to shuttle the desired bifunctional organophosphatase-BBB receptor fusion antibody from peripheral blood into the CNS.

### IV. ORGANOPHOSPHATASE FUSION ANTIBODIES FOR TRANSPORT ACROSS THE BBB

In one aspect, the invention provides compositions and methods that utilize an organophosphatase (e.g., a human PON1) fused to an antibody capable of crossing the BBB. The compositions and methods are useful in transporting an organophosphatase from the peripheral blood and across the BBB into the CNS.

The BBB has been shown to have specific receptors that allow the transport from the blood to the brain of several macromolecules; these transporters are suitable as transporters for compositions of the invention. Endogenous BBB receptor-mediated transport systems useful in the invention include, but are not limited to, those that transport insulin, transferrin, insulin-like growth factors 1 and 2 (IGF1 and IGF2), leptin, and lipoproteins. In some embodiments, the invention utilizes an antibody that is capable of crossing the BBB via the endogenous insulin BBB receptor-mediated transport system, e.g., the human endogenous insulin BBB receptor-mediated transport system. In some embodiments, the organophosphatase fusion antibody comprises an HIR antibody. The organophosphatase-HIRAb fusion antibodies described herein bind to the extracellular domain (ECD) of the human insulin receptor. In some embodiments, the organophosphatase is fused to the C-terminus of the heavy chain immunoglobulin of the HIRAb. In other embodiments, the organophosphatase is fused to the C-terminus of the light chain immunoglobulin of the HIRAb.

Insulin receptors and their extracellular, insulin binding domain (ECD) have been extensively characterized in the art both structurally and functionally. See, e.g., Yip et al., (2003), J. Biol. Chem., 278(30):27329-27332; and Whittaker et al., (2005), J. Biol. Chem., 280(22):20932-20936. The amino acid and nucleotide sequences of the human insulin receptor can be found under GenBank accession No. NM_{—}000208.

Insulin receptors expressed on the BBB can thereby serve as a vector for transport of an organophosphatase, e.g., a human PON1, across the BBB. Certain ECD-specific antibodies may mimic the endogenous ligand and thereby traverse a plasma membrane barrier via transport on the specific receptor system. In certain embodiments, an organophosphatase-HIRAb fusion antibody binds an exofacial epitope on the human BBB HIR and this binding enables the fusion antibody to traverse the BBB via a transport reaction that is mediated by the human BBB insulin receptor.

For use in humans, a chimeric HIR Ab is preferred that contains enough human sequence that it is not significantly immunogenic when administered to humans, e.g., about 80% human and about 20% mouse, or about 85% human and about 15% mouse, or about 90% human and about 10% mouse, or about 95% human and 5% mouse, or greater than about 95% human and less than about 5% mouse. Chimeric antibodies to the human BBB insulin receptor with sufficient human sequences for use in the invention are described in, e.g., Boado et al., (2007), Biotechnol Bioeng, 96(2):381-391. A more highly humanized form of the HIRAb can also be engineered, and the humanized HIR Ab has activity comparable to the murine HIR Ab and can be used in embodiments of the invention. See, e.g., U.S. Patent Application Publication Nos. 20040101904, filed Nov. 27, 2002 and 20050142141, filed Feb. 17, 2005.

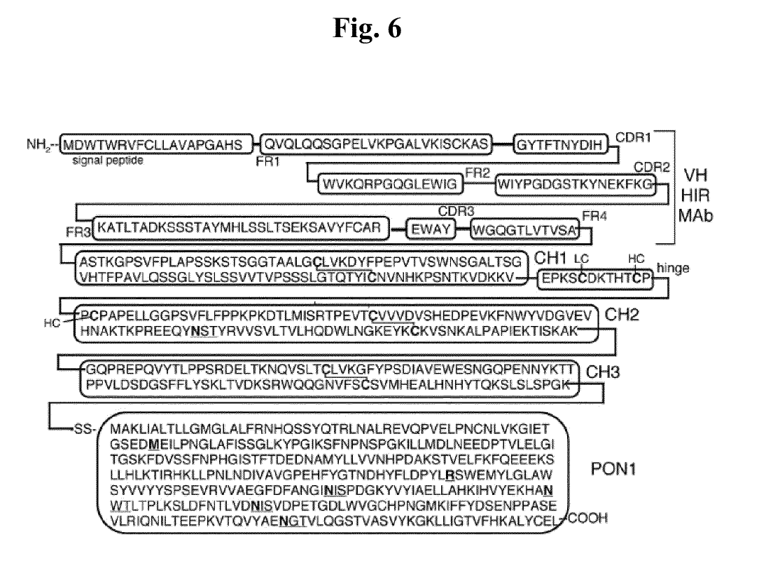

In exemplary embodiments, the organophosphatase-HIR-Ab fusion antibodies contain an immunoglobulin HC comprising CDRs corresponding to the sequence of at least one of the HC CDRs listed in FIG. 6 (SEQ ID NO:21), i.e., CDR 1 (SEQ ID NO:21 amino acids 45-54), CDR2 (SEQ ID NO:21 amino acids 69-85), or CDR3 (SEQ ID NO:21 amino acids 118-121), or variants thereof. For example, the HC can include a HC CDR 1 corresponding to SEQ ID NO:21 amino acids 45-54 with up to 1, 2, 3, 4, 5, or 6 single amino acid mutations, a HC CDR2 corresponding to the amino acid sequence of SEQ ID NO:21 amino acids 69-85 with up to 1, 2, 3, 4, 5, 6, 7, 8, 9, or 10 single amino acid mutations, or a HC CDR3 corresponding to the amino acid sequence of SEQ ID NO:21 amino acids 118-121 with up to 1, or 2 single amino acid mutations, where the single amino acid mutations are substitutions, deletions, or insertions.

In other embodiments, the organophosphatase-HIRAb fusion Abs contain an immunoglobulin HC the amino acid sequence of which is at least 50% identical (i.e., at least, 55, 60, 65, 70, 75, 80, 85, 90, 95, or any other percent up to 100% identical) to amino acids 1-464 of SEQ ID NO:21.

In some embodiments, the organophosphatase-HIRAb fusion Ab competes for binding to the human insulin receptor with an antibody comprising a heavy chain containing amino acids 20-462 of SEQ ID NO:21. In another embodiment, the organophosphatase-HIRAb fusion Ab competes for binding to the human insulin receptor with a fusion antibody comprising the amino acid sequence of SEQ ID NO:23.

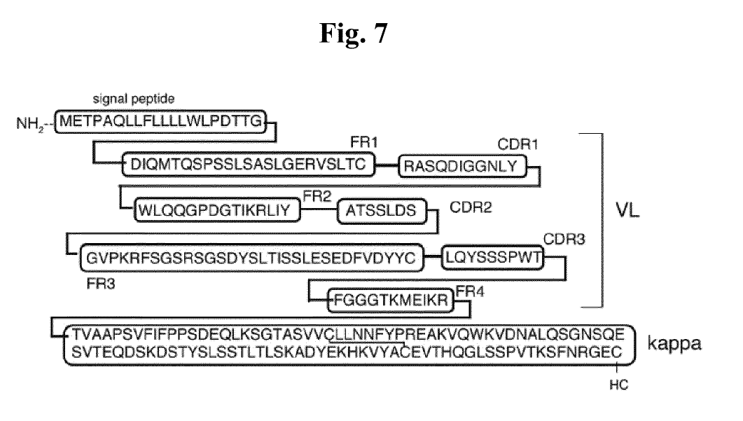

In some embodiments, the HIR Abs or organophosphatase-HIRAb fusion Abs include an immunoglobulin light chain comprising CDRs corresponding to the sequence of at least one of the LC CDRs listed in FIG. 7 (SEQ ID NO:27), i.e. CDR1 (SEQ ID NO:27 amino acids 44-54), CDR2 (SEQ ID NO:27 amino acids 70-76, or CDR3 (SEQ ID NO:27 amino acids 109-117) or a variant thereof. For example, a LC CDR1 corresponding to the amino acid sequence of SEQ ID NO:27 amino acids 44-54 with up to 1, 2, 3, 4, or 5 single amino acid mutations, a LC CDR2 corresponding to the amino acid sequence of SEQ ID NO:27 amino acids 70-76 with up to 1, 2, 3, or 4 single amino acid mutations, or a LC CDR3 corresponding to the amino acid sequence of SEQ ID NO:27 amino acids 109-117 with up to 1, 2, 3, 4, or 5 single amino acid mutations.

In other embodiments, the HIR Abs or organophosphatase HIR-Ab fusion Abs contain an immunoglobulin LC the amino acid sequence of which is at least 50% identical (i.e., at least, 55, 60, 65, 70, 75, 80, 85, 90, 95, or any other percent up to 100% identical) to SEQ ID NO:27 (shown in FIG. 7).

In yet other embodiment, the organophosphatase-HIR-Ab fusion Abs contain both a heavy chain and a light chain corresponding to any of the above-mentioned HIR heavy chains and HIR light chains.

HIR antibodies used in the invention may be glycosylated or non-glycosylated. If the antibody is glycosylated, any pattern of glycosylation that does not significantly affect the function of the antibody may be used. Glycosylation can occur in the pattern typical of the cell in which the antibody is made, and may vary from cell type to cell type. For example, the glycosylation pattern of a monoclonal antibody produced by a mouse myeloma cell can be different than the glycosylation pattern of a monoclonal antibody produced by a transfected Chinese hamster ovary (CHO) cell. In some embodiments, the antibody is glycosylated in the pattern produced by a transfected Chinese hamster ovary (CHO) cell.

One of ordinary skill in the art will appreciate that current technologies permit a vast number of sequence variants of candidate HIR Abs or known HIR Abs to be readily generated (e.g., in vitro) and screened for binding to a target antigen such as the ECD of the human insulin receptor or an isolated epitope thereof. See, e.g., Fukuda et al., (2006), Nuc. Acid Res., 34(19) (published online) for an example of μltra high throughput screening of antibody sequence variants. See also, Chen et al., (1999), Prot Eng, 12(4): 349-356. An insulin receptor ECD can be purified as described in, e.g., Coloma et al., (2000) Pharm Res, 17:266-274, and used to screen for HIR Abs and HIR Ab sequence variants of known HIR Abs.

Accordingly, in some embodiments, a genetically engineered HIR Ab, with the desired level of human sequences, is fused to an organophosphatase, to produce a recombinant fusion antibody that is a bi-functional molecule. The organophosphatase HIR-Ab fusion Ab: (i) binds to an extracellular domain of the HIR; (ii) has organophosphatase activity; and (iii) is able to cross the BBB, via transport on the BBB HIR, and retain organophosphatase activity once inside the brain, following peripheral administration.

As used herein, an organophosphatase refers to any naturally occurring or artificial enzyme that can catalyze the hydrolysis of an organophosphate. Examples of organophosphatases include, but are not limited to, human PON1 (NM_{—}000446; SEQ ID NO: 17), sequence variants of human PON1, and sequence variants of butyrylcholinesterase (BCE), e.g., a human G117H mutant of BCE.

In some embodiments, the organophosphatase in an organophosphatase-BBB receptor Ab fusion antibody (e.g., an organophosphatase-HIRAb fusion antibody) has an amino acid sequence that is a at least 50% identical (i.e., at least, 55, 60, 65, 70, 75, 80, 85, 90, 95, or another percent up to 100% identical) to the amino acid sequence of SEQ ID NO:17 (GenBank Accession No. NM_{—}000446). The structure-function relationship of human PON1 is described in, e.g., Josse et al., (1999), Biochemistry, 38(9):2816-2825 and Josse et al., (2001), J Appl Toxicol, Suppl 1:S7-11.

In particular, residues that are critical to the organophosphatase enzymatic activity of human PON1 include, e.g., C41, C352, D53, D168, D182, D268, D278, E52, E194, H114, H133, H154, H242, H284, and W280.

In some embodiments, the organophosphatase has an amino acid sequence at least 50% identical (i.e., at least, 55, 60, 65, 70, 75, 80, 85, 90, 95, or another percent up to 100% identical) to SEQ ID NO:17. In some embodiments, the organophosphatase in an organophosphatase-BBB receptor Ab fusion antibody is a sequence variant of human PON1 comprising a Met⁵⁵→Leu⁵⁵ substitution (SEQ ID NO:19). Sequence variants of an organophosphatase amino acid sequence, e.g., variants of SEQ ID NOs:17 and 19 can be generated, e.g., by random mutagenesis of the entire sequence or specific subsequences corresponding to particular domains. Alternatively, site directed mutagenesis can be performed reiteratively while avoiding mutations to residues known to be critical to organophosphatase enzymatic activity such as those given above. Further, in generating multiple variants of an organophosphatase sequence such as that of human PON1 or a human PON1 sequence variant, mutation tolerance prediction programs can be used to greatly reduce the number of non-functional sequence variants that would be generated by strictly random mutagenesis. Various programs for predicting the effects of amino acid substitutions in a protein sequence on protein function (e.g., SIFT, PolyPhen, PANTHER PSEC, PMUT, and TopoSNP) are described in, e.g., Henikoff et al., (2006), Annu. Rev. Genomics Hum. Genet., 7:61-80. Organophosphatase sequence variants can be screened for organophosphatase activity using fluorometric organophosphatase assays known in the art. See, e.g., Soukharev et al., (2004), Analytical Biochem, 327(1):140-148, which describes a fluorometric enzyme assay using diethylphospho-6,8-difluoro-4-methyl-umbelliferone (DEPFMU) as the substrate. Accordingly, one of ordinary skill in the art will appreciate that a very large number of operable organophosphatase sequence variants can be obtained by generating and screening extremely diverse “libraries” of organophosphatase (e.g., human PON1, SEQ ID NO:17, and SEQ ID NO:19) sequence variants by methods that are routine in the art, as described above. In some embodiments, such libraries are generated so as to avoid mutations in the above-mentioned residues critical to PON1 enzyme activity). In other embodiments, such libraries include variations throughout the entire sequence.

Percent sequence identity is determined by conventional methods. See, for example, Altschul et al., (1986), Bull. Math. Bio., 48:603, and Henikoff and Henikoff, (1992), Proc. Natl. Acad. Sci. USA, 89:10915. Briefly, two amino acid sequences are aligned to optimize the alignment scores using a gap opening penalty of 10, a gap extension penalty of 1, and the “BLOSUM62” scoring matrix of Henikoff and Henikoff (supra). The percent identity is then calculated as: ([Total number of identical matches]/[length of the longer sequence plus the number of gaps introduced into the longer sequence in order to align the two sequences])(100).

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of another peptide. The FASTA algorithm is described by Pearson et al., (1988), Proc. Nat'l Acad. Sci. USA, 85:2444, and by Pearson (1990), Meth. Enzymol. 183:63. Briefly, FASTA first characterizes sequence similarity by identifying regions shared by the query sequence (e.g., SEQ ID NO:17 or SEQ ID NO: 19) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman et al., (1970), J. Mol. Biol. 48:444; Sellers (1974), SIAM J. Appl. Math., 26:787, which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, (1990), Meth. Enzymol., 183:63.

The present invention also includes proteins having a conservative amino acid change, compared with an amino acid sequence disclosed herein. Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff et al., (1992), Proc. Nat'l Acad. Sci., USA, 89:10915. Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

It also will be understood that amino acid sequences may include additional residues, such as additional N- or C-terminal amino acids, and yet still be essentially as set forth in one of the sequences disclosed herein, so long as the sequence retains sufficient biological protein activity to be functional in the compositions and methods of the invention.

### V. COMPOSITIONS

Strikingly, it has been found that the bifunctional organophosphatase-BBB receptor Ab fusion antibodies described herein retain a high proportion of the activity of their separate constituent proteins, i.e., binding of the BBB receptor Ab to the BBB receptor ECD, e.g., the human insulin receptor ECD, and the enzymatic activity of organophosphatase (e.g., PON1).

Described herein are bifunctional organophosphatase-BBB receptor fusion antibodies containing a BBB receptor ECD Ab capable of crossing the BBB fused to an organophosphatase, where the BBB receptor Ab capable of crossing the blood brain barrier and the organophosphatase each retain an average of at least about 5, 10, 15, 18, 20, 25, 30, 35, 40, 40, 45, or 50% of their activities, compared to their activities as separate entities. In some embodiments, the invention provides a organophosphatase-BBB receptor Ab fusion antibody where the BBB receptor Ab and organophosphatase each retain an average of at least about 50% of their activities, compared to their activities as separate entities. In some embodiments, the invention provides a organophosphatase-BBB receptor Ab fusion antibody where the BBB receptor Ab and organophosphatase each retain an average of at least about 60% of their activities, compared to their activities as separate entities. In some embodiments, the invention provides an organophosphatase-BBB receptor Ab fusion antibody where the BBB receptor Ab and organophosphatase each retain an average of at least about 70% of their activities, compared to their activities as separate entities. In some embodiments, the invention provides a organophosphatase-BBB receptor Ab fusion antibody where the BBB receptor Ab and organophosphatase each retain an average of at least about 80% of their activities, compared to their activities as separate entities. In some embodiments, the invention provides a organophosphatase-BBB receptor Ab fusion antibody where the BBB receptor Ab and organophosphatase each retain an average of at least about 90% of their activities, compared to their activities as separate entities. In some embodiments, the BBB receptor Ab retains at least about 10, 20, 30, 40, 50, 60, 70, 80, 90, 95, 99, or 100% of its activity, compared to its activity as a separate entity, and the organophosphatase retains at least about 10, 20, 30, 40, 50, 60, 70, 80, 90, 95, 99, or 100% of its activity, compared to its activity as a separate entity. Accordingly, described herein are compositions containing a bifunctional organophosphatase-BBB receptor Ab fusion antibody capable of crossing the BBB, where the constituent BBB receptor Ab and organophosphatase each retain, as part of the fusion antibody, an average of at least about 10, 20, 30, 40, 50, 60, 70, 80, 90, 95, 99, or 100% of their activities, i.e., BBB receptor binding and organophosphatase activity, respectively, compared to their activities as separate proteins. Examples of BBB receptor Abs include Abs (e.g. monoclonal Abs) against an insulin receptor (e.g., human insulin receptor), transferrin receptor, and a lipoprotein receptor.

In some embodiments the BBB receptor Ab is an antibody to the ECD of the human insulin receptor (HIR). In some embodiments, the organophosphatase is human PON1 or a sequence variant thereof (e.g., SEQ ID NO:19). In the organophosphatase-BBB receptor Ab fusion antibodies described herein, the covalent linkage between the BBB receptor antibody and the organophosphatase may be to the carboxy or amino terminal of the BBB receptor antibody and the amino or carboxy terminal of the organophosphatase as long as the linkage allows the organophosphatase-BBB receptor Ab fusion antibody to bind to the ECD of the BBB receptor and cross the blood brain barrier, and allows the organophosphatase enzyme to retain a therapeutically useful portion of its activity. In certain embodiments, the covalent link is between a HC of the antibody and the organophosphatase or a LC of the antibody and the organophosphatase. Any suitable linkage may be used, e.g., carboxy terminus of light chain to amino terminus of organophosphatase, carboxy terminus of heavy chain to amino terminus of organophosphatase, amino terminus of light chain to amino terminus of organophosphatase, amino terminus of heavy chain to amino terminus of organophosphatase, carboxy terminus of light chain to carboxy terminus of organophosphatase, carboxy terminus of heavy chain to carboxy terminus of organophosphatase, amino terminus of light chain to carboxy terminus of organophosphatase, or amino terminus of heavy chain to carboxy terminus of organophosphatase. In preferred embodiments, the linkage is from the carboxy terminus of the HC to the amino terminus of the organophosphatase. In some embodiments, the fusion antibody composition comprises a human PON1 organophosphatase covalently linked via its N-terminus to the C-terminus of the heavy chain of a human insulin receptor antibody.

It will be appreciated that a linkage between terminal amino acids can be accomplished by an intervening peptide linker sequence that forms part of the fused amino acid sequence. The peptide sequence linker may be 1, 2, 3, 4, 5, 6, 7, 8, 9, 10, or more than 10 amino acids in length. In some embodiments, a two amino acid linker is used. In some embodiments, the linker has the sequence ser-ser. The peptide linker sequence may include a protease cleavage site, however this is not a requirement for activity of the organophosphatase. Indeed, an advantage of these embodiments of the present invention is that the bifunctional organophosphatase-BBB receptor antibody fusion antibody, without cleavage, is partially or fully active both for transport and for activity once across the BBB. FIG. 6 shows an exemplary embodiment of the amino acid sequence of an organophosphatase-BBB receptor antibody fusion antibody, which is a human PON1-human insulin receptor antibody fusion antibody (SEQ ID NO:21) in which the HC is fused through its carboxy terminus via a two amino acid “ser-ser” linker to the amino terminus of the human PON1 organophosphatase sequence.

In some embodiments, an organophosphatase-BBB receptor Ab fusion antibody comprises both a HC and a LC. In some embodiments, the organophosphatase-BBB receptor Ab fusion antibody is a monovalent antibody. In other embodiments, the organophosphatase-BBB receptor Ab fusion antibody is a divalent antibody, as described herein in the Example section.

The BBB receptor Ab used as part of the organophosphatase-BBB receptor Ab fusion antibody can be glycosylated or nonglycosylated; in some embodiments, the antibody is glycosylated, e.g., in a glycosylation pattern produced by its synthesis in a CHO cell.

As used herein, “activity” includes physiological activity (e.g., ability to cross the BBB and/or therapeutic activity), binding affinity of the BBB receptor Ab for the ECD of its receptor target antigen, or the enzymatic activity of an organophosphatase.

Transport of an organophosphatase-BBB receptor Ab fusion antibody fusion antibody across the BBB may be compared to transport across the BBB of the BBB receptor Ab alone by standard methods. For example, pharmacokinetics and brain uptake of the organophosphatase-BBB receptor Ab fusion antibody by a model animal, e.g., a mammal such as a non-human primate, may be used. Similarly, standard models for determining organophosphatase activity may also be used to compare the function of the organophosphatase alone and as part of an organophosphatase-BBB receptor Ab fusion antibody. See, e.g., Example 5, which demonstrates the enzymatic activity of human PON1 versus human PON1-human insulin receptor Ab fusion antibody. Binding affinity for the BBB receptor ECD can be compared for the organophosphatase-BBB receptor Ab fusion antibody versus the BBB receptor Ab alone. See, e.g., Example 7 herein.

Also included herein are pharmaceutical compositions that contain one or more organophosphatase-BBB receptor Ab fusion antibodies described herein and a pharmaceutically acceptable excipient. A thorough discussion of pharmaceutically acceptable carriers/excipients can be found in Remington's Pharmaceutical Sciences, Gennaro, A R, ed., 20th edition, 2000: Williams and Wilkins P A, USA. Pharmaceutical compositions of the invention include compositions suitable for administration via any peripheral route, including intravenous, subcutaneous, intramuscular, intraperitoneal injection; oral, rectal, transbuccal, pulmonary, transdermal, intranasal, or any other suitable route of peripheral administration.

The compositions of the invention are particularly suited for injection, e.g., as a pharmaceutical composition for intravenous, subcutaneous, intramuscular, or intraperitoneal administration. Aqueous compositions of the present invention comprise an effective amount of a composition of the present invention, which may be dissolved or dispersed in a pharmaceutically acceptable carrier or aqueous medium. The phrases “pharmaceutically” or “pharmacologically acceptable” refer to molecular entities and compositions that do not produce an adverse, allergic or other untoward reaction when administered to an animal, e.g., a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

Exemplary pharmaceutically acceptable carriers for injectable compositions can include calcium salts, for example, such as calcium chlorides, calcium bromides, calcium sulfates, and the like; and the salts of organic acids such as acetates, propionates, malonates, benzoates, and the like. For example, compositions of the invention may be provided in liquid form, and formulated in saline based aqueous solution of varying pH (5-8), with or without detergents such polysorbate-80 at 0.01-1%, or carbohydrate additives, such mannitol, sorbitol, or trehalose. Commonly used buffers include histidine, acetate, phosphate, or citrate. Under ordinary conditions of storage and use, these preparations can contain a preservative to prevent the growth of microorganisms. The prevention of the action of microorganisms can be brought about by various antibacterial and antifungal agents, for example, parabens, chlorobutanol; phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate, and gelatin.

For human administration, preparations meet sterility, pyrogenicity, general safety, and purity standards as required by FDA and other regulatory agency standards. The active compounds will generally be formulated for parenteral administration, e.g., formulated for injection via the intravenous, intramuscular, subcutaneous, intralesional, or intraperitoneal routes. The preparation of an aqueous composition that contains an active component or ingredient will be known to those of skill in the art in light of the present disclosure. Typically, such compositions can be prepared as injectables, either as liquid solutions or suspensions; solid forms suitable for use in preparing solutions or suspensions upon the addition of a liquid prior to injection can also be prepared; and the preparations can also be emulsified.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, methods of preparation include vacuum-drying and freeze-drying techniques which yield a powder of the active ingredient plus any additional desired ingredient from a previously sterile-filtered solution thereof.

Upon formulation, solutions will be systemically administered in a manner compatible with the dosage formulation and in such amount as is therapeutically effective based on the criteria described herein. The formulations are easily administered in a variety of dosage forms, such as the type of injectable solutions described above, but drug release capsules and the like can also be employed

The appropriate quantity of a pharmaceutical composition to be administered, the number of treatments, and unit dose will vary according to the CNS uptake characteristics of an organophosphatase-BBB receptor Ab fusion antibody as described herein, and according to the subject to be treated, the state of the subject, and the dose of organophosphate to which the subject is exposed. The person responsible for administration will, in any event, determine the appropriate dose for the individual subject.

In addition to the compounds formulated for parenteral administration, such as intravenous or intramuscular injection, other alternative methods of administration of the present invention may also be used, including but not limited to intradermal administration (See U.S. Pat. Nos. 5,997,501; 5,848,991; and 5,527,288), pulmonary administration (See U.S. Pat. Nos. 6,361,760; 6,060,069; and 6,041,775), buccal administration (See U.S. Pat. Nos. 6,375,975; and 6,284,262), transdermal administration (See U.S. Pat. Nos. 6,348,210; and 6,322,808) and transmucosal administration (See U.S. Pat. No. 5,656,284). Such methods of administration are well known in the art. One may also use intranasal administration of the present invention, such as with nasal solutions or sprays, aerosols or inhalants. Nasal solutions are usually aqueous solutions designed to be administered to the nasal passages in drops or sprays. Nasal solutions are prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known and include, for example, antibiotics and antihistamines and are used for asthma prophylaxis.

Additional formulations, which are suitable for other modes of administration, include suppositories and pessaries. A rectal pessary or suppository may also be used. Suppositories are solid dosage forms of various weights and shapes, usually medicated, for insertion into the rectum or the urethra. After insertion, suppositories soften, melt or dissolve in the cavity fluids. For suppositories, traditional binders and carriers generally include, for example, polyalkylene glycols or triglycerides; such suppositories may be formed from mixtures containing the active ingredient in any suitable range, e.g., in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations, or powders. In certain defined embodiments, oral pharmaceutical compositions will comprise an inert diluent or assimilable edible carrier, or they may be enclosed in a hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations can contain at least 0.1% of active compound. The percentage of the compositions and preparations may, of course, be varied, and may conveniently be between about 2 to about 75% of the weight of the unit, or between about 25-60%. The amount of active compounds in such therapeutically useful compositions is such that a suitable dosage will be obtained.

The tablets, troches, pills, capsules and the like may also contain the following: a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent, methylene and propyl parabens as preservatives, a dye and flavoring, such as cherry or orange flavor. In some embodiments, an oral pharmaceutical composition may be enterically coated to protect the active ingredients from the environment of the stomach; enteric coating methods and formulations are well-known in the art.

### VI. NUCLEIC ACIDS, VECTORS, AND MANUFACTURE

The invention also provides nucleic acids, vectors, cells, and methods of production. In some embodiments, the invention provides nucleic acids that code for polypeptides described herein, e.g., an organophosphatase fused to the HC of a BBB receptor Ab, or an organophosphatase fused to the LC of a BBB receptor Ab. In certain embodiments, the invention provides a single nucleic acid sequence containing a first sequence coding for a light chain of an immunoglobulin and a second sequence coding a heavy chain of the immunoglobulin, where either the first sequence also codes for an organophosphatase that is expressed as a fusion protein with the light chain, or the second sequence also codes for the organophosphatase that is expressed as a fusion protein with the heavy chain. In some embodiments, the invention provides nucleic acid sequences, and in some embodiments the invention provides nucleic acid sequences that are at least about 60, 70, 80, 90, 95, 99, or 100% identical to a particular nucleotide sequence. For example, in some embodiments, the invention provides a nucleic acid containing a first sequence that is at least about 60, 70, 80, 90, 95, 99, or 100% identical to nucleotides 750-3209 of SEQ ID NO:30 and a second sequence that is at least about 60, 70, 80, 90, 95, 99, or 100% identical to nucleotides 4277-4981 of SEQ ID NO:30.

For sequence comparison, of two nucleic acids, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, including but not limited to, by the local homology algorithm of Smith and Waterman, (1970), Adv. Appl. Math., 2:482c, by the homology alignment algorithm of Needleman and Wunsch, (1970), J. Mol. Biol., 48:443, by the search for similarity method of Pearson and Lipman, (1988), Proc. Nat'l. Acad. Sci. USA, 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., (1995 supplement), Current Protocols in Molecular Biology).

One example of an algorithm that is suitable for determining percent sequence identity and sequence similarity are the BLAST and BLAST 2.0 algorithms, which are described in Altschul et al., (1977), Nuc. Acids Res., 25:3389-3402, and Altschul et al., (1990), J. Mol. Biol., 215:403-410, respectively. Software for performing BLAST analyses is publicly available through the National Center for Biotechnology Information. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. The BLAST algorithm is typically performed with the “low complexity” filter turned off. The BLAST algorithm also performs a statistical analysis of the similarity between two sequences (see, e.g., Karlin and Altschul, (1993), Proc. Natl. Acad. Sci. USA, 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The invention provides nucleic acids that code for any of the polypeptides (e.g., fusion antibodies) of the invention. In some embodiments, the invention provides a single nucleic acid sequence containing a gene coding for a light chain of an immunoglobulin and a gene coding for a fusion protein, where the fusion protein includes a heavy chain of the immunoglobulin covalently linked to an organophosphatase. In other embodiments, the invention provides a single nucleic acid sequence containing a gene coding for a heavy chain of an immunoglobulin and a gene coding for a fusion protein, where the fusion protein includes a light chain of the immunoglobulin covalently linked to an organophosphatase. In some embodiments, the organophosphatase is a human PON1 or a sequence variant thereof, as described herein. The antibody can be an antibody to a transport system, e.g., an endogenous BBB receptor-mediated transport system such as the endogenous BBB receptor-mediated insulin transport system. In some embodiments, the endogenous BBB receptor-mediated insulin transport system is a human endogenous BBB receptor-mediated insulin transport system and wherein the organophosphatse to which the encoded antibody heavy chain is covalently linked is human PON1. Any suitable organophosphatase, antibody, monoclonal antibody, or chimeric antibody, as described herein, may be coded for by the nucleic acid, combined as a fusion protein and coded for in a single nucleic acid sequence. As is well-known in the art, owing to the degeneracy of the genetic code, any combination of suitable codons may be used to code for the desired fusion protein. In addition, other elements useful in recombinant technology, such as promoters, termination signals, and the like, may also be included in the nucleic acid sequence. Such elements are well-known in the art. In addition, all nucleic acid sequences described and claimed herein include the complement of the sequence.

In some embodiments of a nucleic acid that encodes an organophosphatase, e.g., a variant organophosphatase, as a component of the fusion antibodies described herein, the encoded organophosphatase contains an amino acid sequence that is about 60, 70, 80, 90, 95, 99, or 100% identical to the sequence SEQ ID NOs 17 or 19. In some embodiments, the encoded organophosphatase is linked at its amino terminus to carboxy terminus of the heavy chain of the BBB receptor Ab, e.g., a MAb HC. The heavy chain of the MAb can comprise a sequence that is about 60, 70, 80, 90, 95, 99 or 100% identical to amino acids 20-462 of SEQ ID NOs: 21 or 23. In some embodiments, a nucleic acid encodes a light chain of the BBB receptor Ab, e.g., MAb, comprising an encoded amino acid sequence that is about 60, 70, 80, 90, 95, 99 or 100% identical to amino acids 21-234 of SEQ ID NO: 27. The nucleic acid can further contain a nucleic acid sequence that codes for a peptide linker between the heavy chain of the MAb and the organophosphatase. In some embodiments, the linker is S-S-M. In other embodiments, the linker is S-S. In another embodiment, the linker is S-S-S. The nucleic acid may further contain a nucleic acid sequence coding for a signal peptide, wherein the signal peptide is linked to the heavy chain. Any suitable signal peptide, as known in the art or subsequently developed, may be used. In some embodiments, the signal peptide attached to the heavy chain comprises a sequence that is about 60, 70, 80, 90, 95, 99, or 100% identical to amino acids 1-19 of SEQ ID NO: 21. In some embodiments, the nucleic acid contains a nucleic acid sequence coding for another signal peptide, wherein the other signal peptide is linked to the light chain. The signal peptide linked to the light chain can comprise a sequence that is about 60, 70, 80, 90, 95, 99, or 100% identical to amino acids 1-20 of SEQ ID NO: 27. The nucleic acid can contain a nucleic acid sequence coding for a selectable marker. In some embodiments the selectable marker is DHFR. In some embodiments, the encoded sequence of the DHFR can be about 60, 70, 80, 90, 95, 99, or 100% identical to amino acids 1-187 of SEQ ID NO: 33.

In certain embodiments, the invention provides a nucleic acid comprising a first sequence that codes for an organophosphatase, e.g., a neurotrophin such as human PON1, in the same open reading frame as a second sequence that codes for an immunoglobulin component. The immunoglobulin component can be, e.g., a heavy chain, e.g., that is at least about 60, 70, 80, 90, 95, 99, or 100% identical to nucleotides 750-2135 of SEQ ID NO: 30 and a second (light chain) sequence that is at least about 60, 70, 80, 90, 95, 99, or 100% identical to nucleotides 4277-4981 of SEQ ID NO: 30. In some embodiments, the nucleic acid also contains a third sequence that encodes for a selectable marker such as DHFR.

In some embodiments, nucleic acids of the invention hybridize specifically under low, medium, or high stringency conditions to a nucleic acid encoding the amino acid sequence of SEQ ID NO:23, or hybridize to the complement of a nucleic acid encoding SEQ ID NO:22. Low stringency hybridization conditions include, e.g., hybridization with a 100 nucleotide probe of about 40% to about 70% GC content at 42° C. in 2×SSC and 0.1% SDS. Medium stringency hybridization conditions include, e.g., at 50° C. in 0.5×SSC and 0.1% SDS. High stringency hybridization conditions include, e.g., hybridization with the above-mentioned probe at 65° C. in 0.2×SSC and 0.1% SDS. Under these conditions, as the hybridization temperature is elevated, a nucleic acid with a higher homology can be obtained.

The invention also provides vectors. The vector can contain any of the nucleic acid sequences described herein. In some embodiments, the invention provides a single tandem expression vector containing nucleic acid coding for an antibody heavy chain fused to an organophosphatase, e.g., a human PON1 or sequence variant thereof, and nucleic acid coding for a light chain of the antibody, all incorporated into a single piece of nucleic acid, e.g., a single piece of DNA referred to herein as a “tandem vector.” The single tandem vector can also include one or more selection and/or amplification genes. A method of making an exemplary vector of the invention is provided in the Examples. However, any suitable techniques, as known in the art, may be used to construct the vector.

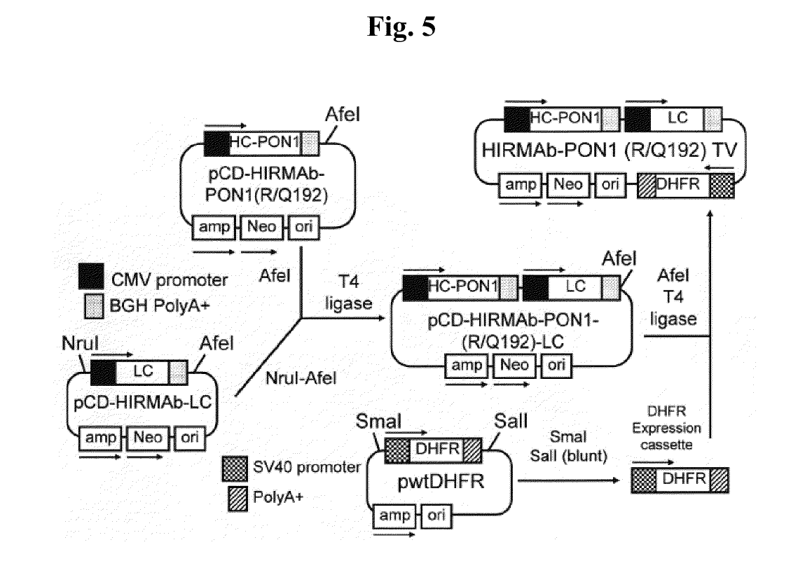

The use of a single tandem vector has several advantages. The transfection of a eukaryotic cell line with immunoglobulin G (IgG) genes generally involves the co-transfection of the cell line with separate plasmids encoding the heavy chain (HC) and the light chain (LC) comprising the IgG. In the case of a IgG fusion protein, the gene encoding the recombinant therapeutic protein may be fused to either the HC or LC gene. However, this co-transfection approach makes it difficult to select a cell line that has equally high integration of both the HC and LC-fusion genes, or the HC-fusion and LC genes. The approach to manufacturing the fusion protein utilized in certain embodiments of the invention is the production of a cell line that is permanently transfected with a single plasmid DNA that contains all the required genes on a single strand of DNA, including the HC-fusion protein gene, the LC gene, the selection gene, e.g. neo, and the amplification gene, e.g. the dihydrofolate reductase gene. As shown in the diagram of the fusion protein tandem vector in FIG. 5, the HC-PON1 fusion gene, the LC gene, the neo gene, and the DHFR gene are all under the control of separate, but tandem promoters and separate but tandem transcription termination sequences. Therefore, all genes are equally integrated into the host cell genome, including the fusion gene of the therapeutic protein and either the HC or LC IgG gene.

The invention further provides cells that incorporate one or more of the vectors of the invention. The cell may be a prokaryotic cell or a eukaryotic cell. In some embodiments, the cell is a eukaryotic cell. In some embodiments, the cell is a mouse myeloma hybridoma cell. In some embodiments, the cell is a Chinese hamster ovary (CHO) cell. Exemplary methods for incorporation of the vector(s) into the cell are given in the Examples. However, any suitable techniques, as known in the art, may be used to incorporate the vector(s) into the cell. In some embodiments, the invention provides a cell capable of expressing an immunoglobulin fusion protein, where the cell is a cell into which has been permanently introduced a single tandem expression vector, where both the immunoglobulin light chain gene and the gene for the immunoglobulin heavy chain fused to the therapeutic agent, are incorporated into a single piece of nucleic acid, e.g., DNA. In some embodiments, the invention provides a cell capable of expressing an immunoglobulin fusion protein, where the cell is a cell into which has been permanently introduced a single tandem expression vector, where both the immunoglobulin heavy chain gene and the gene for the immunoglobulin light chain fused to the therapeutic agent, are incorporated into a single piece of nucleic acid, e.g., DNA. The introduction of the tandem vector may be by, e.g., integration into the chromosomal nucleic acid, or by, e.g., introduction of an episomal genetic element.

In addition, the invention provides methods of manufacture. In some embodiments, the invention provides a method of manufacturing an immunoglobulin fusion protein, where the fusion protein contains an immunoglobulin heavy chain fused to an organophosphatase, by permanently introducing into a eukaryotic cell a single tandem expression vector, where both the immunoglobulin light chain gene and the gene for the immunoglobulin heavy chain fused to the organophosphatase, are incorporated into a single piece of nucleic acid, e.g., DNA. In some embodiments, the invention provides a method of manufacturing an immunoglobulin fusion protein, where the fusion protein contains an immunoglobulin light chain fused to an organophosphatase, by introducing into a eukaryotic cell a single tandem expression vector, where both the immunoglobulin heavy chain gene and the gene for the immunoglobulin light chain fused to the organophosphatase, are incorporated into a single piece of nucleic acid, e.g., DNA. In some embodiments, the introduction of the vector is accomplished by integration into the host cell genome. In some embodiments, the introduction of the vector is accomplished by introduction of an episomal genetic element containing the vector into the host cell. Episomal genetic elements are well-known in the art. In some embodiments, the organophosphatase is a human PON1 or a sequence variant thereof. In some embodiments, the single piece of nucleic acid further includes one or more genes for selectable markers. In some embodiments, the single piece of nucleic acid further includes one or more amplification genes. In some embodiments, the immunoglobulin is an IgG, e.g., a MAb such as a chimeric MAb. The methods may further include expressing the immunoglobulin fusion protein, and/or purifying the immunoglobulin fusion protein. Exemplary methods for manufacture, including expression and purification, are given in the Examples.

However, any suitable technique, as known in the art, may be used to manufacture, optionally express, and purify the proteins. These include non-recombinant techniques of protein synthesis, such as solid phase synthesis, manual or automated, as first developed by Merrifield and described by Stewart et al., (1984) in Solid Phase Peptide Synthesis. Chemical synthesis joins the amino acids in the predetermined sequence starting at the C-terminus. Basic solid phase methods require coupling the C-terminal protected α-amino acid to a suitable insoluble resin support Amino acids for synthesis require protection on the α-amino group to ensure proper peptide bond formation with the preceding residue (or resin support). Following completion of the condensation reaction at the carboxyl end, the α-amino protecting group is removed to allow the addition of the next residue. Several classes of α-protecting groups have been described, see Stewart et al., (1984) in Solid Phase Peptide Synthesis, with the acid labile, urethane-based tertiary-butyloxycarbonyl (Boc) being the historically preferred. Other protecting groups, and the related chemical strategies, may be used, including the base labile 9-fluorenylmethyloxycarbonyl (FMOC). Also, the reactive amino acid sidechain functional groups require blocking until the synthesis is completed. The complex array of functional blocking groups, along with strategies and limitations to their use, have been reviewed by Bodansky (1976) in Peptide Synthesis and, Stewart et al., (1984) in Solid Phase Peptide Synthesis.

Solid phase synthesis is initiated by the coupling of the described C-terminal α-protected amino acid residue. Coupling requires activating agents, such as dicyclohexycarbodiimide (DCC) with or without 1-hydroxybenzo-triazole (HOBT), diisopropylcarbodiimide (DIIPC), or ethyldimethylaminopropylcarbodiimide (EDC). After coupling the C-terminal residue, the α-amino protected group is removed by trifluoroacetic acid (25% or greater) in dichloromethane in the case of acid labile tertiary-butyloxycarbonyl (Boc) groups. A neutralizing step with triethylamine (10%) in dichloro-methane recovers the free amine (versus the salt). After the C-terminal residue is added to the resin, the cycle of deprotection, neutralization and coupling, with intermediate wash steps, is repeated in order to extend the protected peptide chain. Each protected amino acid is introduced in excess (three to five fold) with equimolar amounts of coupling reagent in suitable solvent. Finally, after the completely blocked peptide is assembled on the resin support, reagents are applied to cleave the peptide form the resin and to remove the side chain blocking groups. Anhydrous hydrogen fluoride (HF) cleaves the acid labile tertiary-butyloxycarbonyl (Boc) chemistry groups. Several nucleophilic scavengers, such as dimethylsulfide and anisole, are included to avoid side reactions especially on side chain functional groups.

### VII. METHODS

Described herein are methods for delivering an effective dose of an organophosphatase to the CNS across the BBB by systemically administering a BBB receptor Ab fusion antibody, as described herein. In some embodiments, the compositions described herein are administered to treat organophosphate intoxication, e.g., acute organophosphate intoxication or chronic organophosphate intoxication. In some embodiments, the methods described herein include protecting a subject at high risk of organophosphate intoxication by administering prophylactically the organophosphatase-BBB receptor Ab fusion antibody compositions described herein. Examples of neurotoxic organophosphates that may be treated include, but are not limited to, nerve gases such as Sarin, Tabun, Soman, VX, and pesticides such as parathion, malathion, and azinphosmethyl. In other embodiments, the compositions described herein are administered to treat vascular atherosclerosis.

Suitable systemic doses for delivery of an organophosphatase-BBB receptor Ab fusion antibody will vary based on the organophosphatase specific activity of the organophosphatase-BBB receptor Ab fusion antibody to be administered, and its CNS uptake characteristics, e.g., the percentage of the systemically administered dose to be taken up in the CNS.

In some embodiments, 0.3% (i.e., about 0.32%, 0.4%, 0.48%, 0.6%, 0.74%, 0.8%, 0.9%, 1.05, 1.1, 1.2, 1.3%, 1.5%, 2%, 2.5%, 3%, or another percentage from about 0.3% to about 3%) of the systemically administered organophosphatase-BBB receptor Ab fusion antibody is delivered to the brain as a result of its uptake from peripheral blood across the BBB. In some embodiments, at least 0.5% to at least about 3%, (i.e., about 0.32%, 0.4%, 0.48%, 0.6%, 0.74%, 0.8%, 0.9%, 1.05, 1.1, 1.2, 1.3%, 1.5%, 2%, 2.5%, 3%, or any % from about 0.3% to about 3%) of the systemically administered dose of the organophosphatase-BBB receptor Ab fusion antibody is delivered to the brain within two hours or less, i.e., 1.8, 1.7, 1.5, 1.4, 1.3, 1.2, 1.1, 0.9, 0.8, 0.6, 0.5 or any other period from about 0.25 to about two hours after systemic administration.

Accordingly, in some embodiments the invention provides methods of administering an organophosphatase-BBB receptor Ab fusion antibody systemically, such that the amount of the organophosphatase-BBB receptor Ab fusion antibody to cross the BBB provides at least 00.04 units of organophosphatase activity/mg protein in the subject's brain, i.e., 0.021, 00.22, 0.025, 0.04, 0.05, 0.06, 0.07, 00.9, 0.1, 0.0.11, 0.12, 0.13, 0.14, 0.1.5, 0.16, 0.1.7, 0.2, 0.22, 0.24, 0.25, 0.27, 0.28, 0.3, 0.4, or any other value from 0.004 to 0.4 of units of organophosphatase activity/mg of brain protein of the subject's brain.

In some embodiments, the total number of units of organophosphatase activity delivered to a subject's brain is at least 500 units, i.e., at least 2500, 3000, 3500, 4000, 4000, 5000, 6000, 7000, 8000, 9000, 11000, 12000, 13000, 14000, 20000, 21000, 22000, 23000, 24000, 25000, 26000, 27000, 28000, 30000, 35000, 45000, 47500, or another total number of organophosphatase units from about 500 to 50,000 units of organophosphatase activity.

In some embodiments, a therapeutically effective systemic dose comprises at least 5×10⁴, 1×10⁵, 2×10⁵, 3×10⁵, 4, 105, 5×10⁵, 6×10⁶, 7×10⁵, 8×10⁵, 9×105, 1×10⁶, 1.1×10⁶, 1.2×10⁶, 1.5×10⁶, 1.6×10⁶, 1.7×10⁶, 1.8×10⁶, 1.9×10⁶, 2×10⁶, 2.1×10⁶, 3×10⁶ units of organophosphatase activity, or another systemic dose from about 5×10⁴ to 5×10⁶ units of organophosphatase activity.

In other embodiments, a therapeutically effective systemic dose is at least about 50,000 units of organophosphatase activity/kg body weight, i.e., at least about 6000, 7500, 11000, 12000, 13000, 14000, 20000, 21000, 22000, 23000, 24000, 25000, 26000, 27000, 28000, 30000 or any other number of organophosphatase units from about 100,0 to 100,000 units of organophosphatase activity/kg of body weight.

One of ordinary skill in the art will appreciate that the mass amount of a therapeutically effective systemic dose of an organophosphatase-BBB receptor Ab fusion antibody will depend, in part, on its organophosphatase specific activity. In some embodiments, the organophosphatase specific activity of an organophosphatase-BBB receptor Ab fusion antibody is at least 1000 U/mg of protein, i.e., at least about 1100, 12000, 13000, 14000, 20000, 21000, 22000, 23000, 24000, 25000, 26000, 27000, 28000, 30000, 32000, 34000, 35000, 36000, 37000, 37300, 40000, 100,000, or any other specific activity value from about 100,0 units/mg to about 100,000 units/mg.

Thus, with due consideration of the specific activity of an organophosphatase-BBB receptor Ab fusion antibody and the body weight of a subject to be treated, a systemic dose of the organophosphatase-BBB receptor Ab fusion antibody can be at least 25 mg to about 2500 mg i.e., 50, 100, 150, 200, 250, 300, 350, 400, 450, 500, 550, 600, 650, 700, 750, 1000, 1500, 1750, 2000, 2500, 2750, or any other value from at least about 25 mg to about 2500 mg of organophosphatase-BBB receptor Ab fusion antibody. In some embodiments, the organophosphatase-BBB receptor Ab fusion antibody is a human PON1-human insulin receptor Ab fusion antibody. In some embodiments, the organophosphatase-BBB receptor Ab fusion antibody comprises the amino acid sequence of SEQ NO:21 and SEQ ID NO:27.

The term “systemic administration” or “peripheral administration,” as used herein, includes any method of administration that is not direct administration into the CNS, i.e., that does not involve physical penetration or disruption of the BBB. “Systemic administration” includes, but is not limited to, intravenous, intra-arterial intramuscular, subcutaneous, intraperitoneal, intranasal, transbuccal, transdermal, rectal, transalveolar (inhalation), or oral administration. Any suitable organophosphatase-BBB receptor Ab fusion antibody, as described herein, may be used.

The compositions of the invention may be administered as part of a combination therapy. The combination therapy involves the administration of a composition of the invention in combination with another therapy for treatment or relief of symptoms typically found in a patient suffering from organophosphate intoxication or atherosclerosis. If the composition of the invention is used in combination with another CNS disorder method or composition, any combination of the composition of the invention and the additional method or composition may be used. Thus, for example, if use of a composition of the invention is in combination with another CNS disorder treatment agent, the two may be administered simultaneously, consecutively, in overlapping durations, in similar, the same, or different frequencies, etc. In some cases a composition will be used that contains a composition of the invention in combination with one or more other CNS disorder treatment agents.

In some embodiments, the composition, e.g., a PON1-human insulin receptor Ab fusion antibody is co-administered to the patient with another medication, either within the same formulation or as a separate composition. For example, the organophosphatase fusion antibody could be formulated with another bifunctional fusion antibody that is also designed to deliver across the human blood-brain barrier a recombinant protein other than organophosphatase. Further, the organophosphatase-BBB receptor Ab may be formulated in combination with other large or small molecules.

### EXAMPLES

The following specific examples are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety. Where reference is made to a URL or other such identifier or address, it is understood that such identifiers can change and particular information on the internet can come and go, but equivalent information can be found by searching the internet. Reference thereto evidences the availability and public dissemination of such information.

### Example 1

### Cloning and Expression of the Human PON1 cDNA, and Requirement for Lipid Acceptor in the Medium to Enable PON1 Secretion by Transfected Host Cell

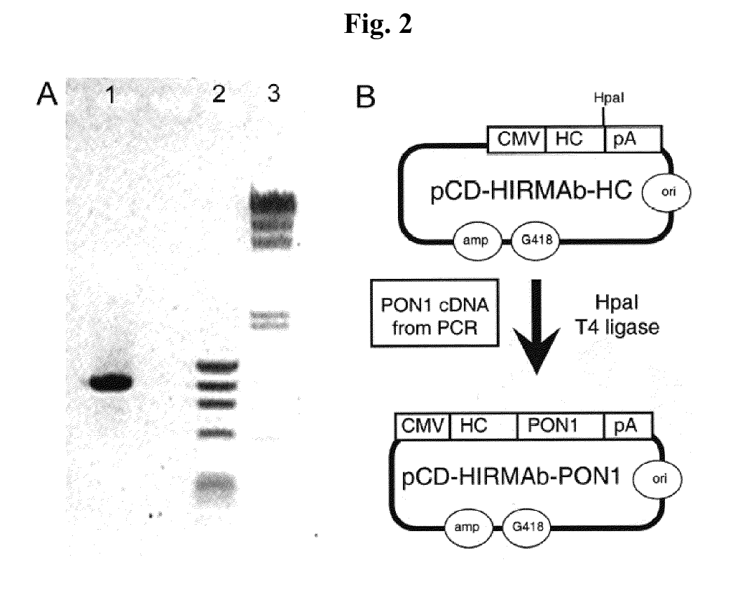

The human PON1 cDNA corresponding to amino acids Met [symbol] 1-Leu355 was cloned by the polymerase chain reaction (PCR) using the oligodexoynucleotides (ODNs) listed in Table 1 and cDNA derived from reverse transcription of human liver PolyA+ RNA. The forward (FWD) ODN primer (SEQ ID NO:1) introduces a Kozak sequence (i.e. CCCGACC) prior to the ATG initiation codon. The reverse ODN primer (SEQ ID NO:2) is complementary to the end of the open reading frame (orf) of PON1 plus 7 nucleotides of the 3′-untranslated region. The PON1 cDNA was cloned by PCR using 25 ng polyA+RNA− derived cDNA, 0.2 μM forward and reverse ODN primers (Table 1), 0.2 mM deoxynucleosidetriphosphates, and 2.5 U PfuUltra DNA polymerase (Stratagene, San Diego, Calif.) in a 50 μl Pfu buffer (Stratagene) containing 7% dimethylsulfoxide. The amplification was performed in a Mastercycler temperature cycler (Eppendorf, Hamburg, Germany) with an initial denaturing step of 95° C. for 2 min followed by 30 cycles of denaturing at 95° C. for 30 sec, annealing at 55° C. for 30 sec and amplification at 72° C. for 1 min; followed by a final incubation at 72° C. for 10 min. PCR products were resolved in 0.8% agarose gel electrophoresis, and the expected major single band of ˜1.1 kb corresponding to the human PON1 cDNA was produced (FIG. 2A). The human PON1 cDNA was subcloned into the EcoRV site of the pcDNA3.1 eukaryotic expression plasmid, which was treated with alkaline phosphatase to prevent self ligation, and this PON1 expression plasmid is designated pCD-PON1. The engineering of this plasmid was validated by DNA sequencing in both directions. The nucleotide sequence of the cloned human PON1 is given in SEQ ID NO:16, and the deduced amino acid sequence of the cloned human PON1 is given in SEQ ID NO:17. There is a 100% match with the known amino acid sequence of human PON1 (GenBank NM_{—}000446). The predicted molecular weight, minus glycosylation, of the PON1 was 39,773 Da with an isoelectric point (pI) of 5.15. The sequence analysis indicated the PCR cloned human PON1 was the Met-55/Arg-192 allozyme. Naturally occurring polymorphisms at these positions include Leu at position 55 and Gln at position 192.

To confirm the biologic activity of the cloned PON1 cDNA, COS cells were transfected with pCD-PON1 using Lipofectamine 2000, with a ratio of 1:2.5, ug DNA:uL Lipofectamine 2000. Following transfection, the cells were cultured in serum free VP-SFM (Invitrogen, Carlsbad, Calif.), with or without 1% Ex-Cyte (Cellianca, Kankakee, Ill.), a lipoprotein supplement. The conditioned serum free medium was collected at 3 and 7 days. PON1 enzyme activity was measured with a fluorometric enzyme assay using diethylphospho-6,8-difluoro-4-methyl-umbelliferone (DEPFMU) as the substrate (Soukharev and Hammond, (2004), Anal Biochem, 327:140-148) which was synthesized by Molecular Probes-Invitrogen (Carlsbad, Calif.). The assay buffer was 0.02 M Tris/pH=8.0/0.15 M NaCl/2 mM CaCl2. The standard curve (3 to 300 pmol/tube) was generated with the reaction product, 6,8-difluoro-4-methylumbelliferone. Human plasma was used as a positive control. Fluorometric readings were obtained with a filter fluorometer using a filter with an emission wavelength of 460 nm and a filter with an excitation wavelength of 355 nm. Enzyme activity is reported as nmol/hour/mL. Enzyme activity was typically measured after both 20 and 40 min incubations, and was always linear with respect to time of incubation. Transfection of COS cells with pCD-PON1 resulted in an increase in PON1 enzyme activity in the medium, and the addition of 1% ExCyte lipid supplement caused a 19-fold increase in medium PON1 enzyme activity (Table 2). The medium PON1 enzyme activity was comparable to PON1 enzyme activity in 10% human plasma (Table 2). These results indicated a functional PON1 cDNA had been cloned, which could now be fused to the cDNA of a BBB molecular Trojan horse, such as the cDNA encoding the heavy or light chain of the HIRMAb.

### Example 2

### M55L Site-Directed Mutagenesis of Human PON1

The cloned human PON1 expressed Met-55 and Arg-192 (SEQ ID NO 17). The more active PON1 allozyme, expressing the Leu-55 polymorphism, was produced with site-directed mutagenesis (SDM). The pCD-PON1 plasmid was used as template to generate the pCD-PON1-Leu55 plasmid. The SDM was performed using the QuickChange II XL SDM kit (Stratagene) and standard protocol. For this M55L SDM, forward and reverse ODNs were used (SEQ ID NOs:3 and 4, respectively); the ODNs were designed to introduce the mutation of interest, i.e. “A” for “T” at position 163 (where position 1 is the beginning of the PON1 orf), and contain 15 nucleotides at each flanking region to anneal with the target sequence. The SDM was verified by bi-directional DNA sequencing. The nucleotide and deduced amino acid sequences for human PON1-Leu55 are shown in SEQ ID NOs:18 and 19, respectively.

### Example 3

### Genetic Engineering of Expression Plasmids Encoding Heavy Chain-PON1 Fusion Protein wherein the PON1 is Fused to Either the Amino Terminus or the Carboxyl Terminus of the HIRMAb Heavy Chain

Owing to the uncertainty as to whether a PON1 fusion protein could be engineered, and still maintain PON1 enzyme activity, the PON1 cDNA was fused to either the 5′-end or the 3′-end of the HIRMAb HC cDNA. In the construct designated PON1-HC, the carboxyl terminus of the PON1 is fused to the amino terminus of the HIRMAb HC. Since PON1 has no signal peptide, this fusion gene was engineered without a leader peptide. This heavy chain fusion protein is expressed by the pCD-PON1-HIRMAb.

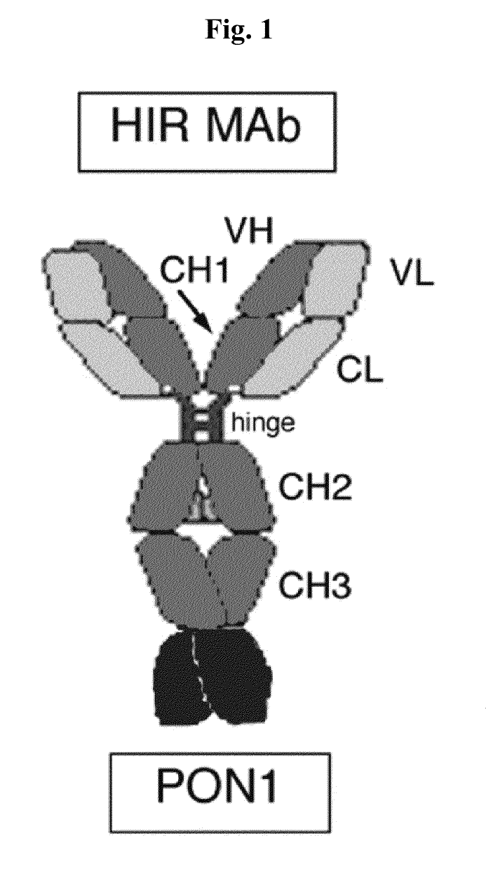

In the construct designated HC-PON1, the carboxyl terminus of the HIRMAb HC is fused to the amino terminus of the PON1, as depicted in FIG. 1. The HC-PON1 included sequence encoding for a 19 amino acid (AA) IgG signal peptide. This heavy chain fusion protein is expressed by the pPON1-HIRMAb (FIG. 2B).

For the engineering of the pCD-HIRMAb-PON1-Met55 plasmid, or the pCD-HIRMAb-PON1-Leu55 plasmid, the mature human PON1-Met55 or -Leu55 cDNA corresponding to amino acids Met [symbol] 1-Leu355 of SEQ ID NO:17, or SEQ ID NO:19, respectively, was cloned by PCR using the pCD-PON1-Met55 or pCD-PON1-Leu55 as template. The PCR cloning reaction was performed as described in Example 1. The ODNs used for PCR are 5′-phosphorylated for direct insertion into the HpaI site of the pCD-HIRMAb-HC expression plasmid (FIG. 2B). The pCD-HIRMAb-HC plasmid encodes the HC of the chimeric HIRMAb, and dual transfection of COS cells with this plasmid and a light chain (LC) expression plasmid, pCD-HIRMAb-LC, allows for transient expression of the chimeric HIRMAb in COS cells. The engineering of the gene encoding the HIRMAb-PON1 fusion protein was performed by ligation of the PCR generated PON1 cDNA (i.e. PON1-Met55 or Leu55) into the single HpaI site of a universal HIRMAb-HC vector (pCD-HIRMAb-HC, FIG. 2B), to form the pCD-HIRMAb-PON1 expression vector (FIG. 2B). Following linearization of the pCD-HIRMAb-HC with HpaI, the plasmid was treated with alkaline phosphatase to prevent self-ligation during the engineering of the pCD-HIRMAb-PON1 expression vector. The engineering of the pCD-HIRMAb-HC was performed by insertion of a single HpaI site at the end of the HIRMAb HC CH3 open reading frame (orf) by site directed mutagenesis. The SDM protocol replaces the TGA stop codon of the HIRMAb HC and the 4 nt on its 3′-flanking region (i.e. TGAGGAT) with the HpaI site with an “A” on its 5′-flanking region (i.e. AGTTAAC). The mature human PON1 forward PCR primer (SEQ ID NO:5) introduces “CA” nucleotides to maintain the open reading frame and to introduce a Ser-Ser linker between the carboxyl terminus of the CH3 region of the HIRMAb HC and the amino terminus of the PON1. The PON1 reverse PCR primer (Table 1, SEQ ID NO:2) introduces a stop codon, “TGA,” immediately after the terminal leucine of the mature PON1 protein. The engineered pCD-HIRMAb-PON1-Met55 or pCD-HIRMAb-PON1-Leu55 expression vectors were validated by DNA sequencing, which confirmed the presence of the cytomegalovirus (CMV) promoter at the 5′-end of the expression cassette, and the bovine growth hormone (BGH) polyA sequence at the 3′-end of the expression cassette. The nucleotide sequence of the HIRMAb-PON1-Met55 HC fusion protein is given in SEQ ID NO:20, and the deduced amino acid sequence is given in SEQ ID NO 21. The nucleotide sequence of the HIRMAb-PON1-Leu55 HC fusion protein is given in SEQ ID NO:22, and the deduced amino acid sequence is given in SEQ ID NO 23. These plasmids encode for a 819 amino acid protein, comprised of a 19 amino acid IgG signal peptide, a 113 amino acid variable region of the heavy chain (VH) of the HIRMAb, a 330 amino acid human IgG1 constant region, a 2 amino acid linker (Ser-Ser), and the 355 amino acid human PON1. The predicted molecular weight of the HIRMAb-PON1-Met55 heavy chain fusion protein, minus glycosylation, is 88,553 Da, with a predicted pI of 6.31; these parameters for the HIRMAb-PON1-Leu55 HC fusion protein are 88,463 and 6.18, respectively.

An expression plasmid was also engineered wherein the carboxyl terminus of the PON1-Leu55 was fused to the amino terminus of the HIRMAb HC, and this plasmid is designated pPON1-HIRMAb-HC. The construction of the pPON1-HIRMAb-HC expression vector was completed with the engineering of an intermediate vector created to introduce an AfeI site that replaced the PON1 stop codon in the pCD-PON1-Leu55 plasmid. The latter was performed by SDM using forward and reverse ODNs (SEQ ID NO:6 and 7, respectively). ODNs were designed to introduce the mutation of interest, i.e. the PON1 stop codon plus 3 nucleotides (TAACAG) were converted into the AfeI site (AGCGCT), and contain 15 nucleotides at each flanking region to anneal with the target sequence. The SDM was verified by bi-directional DNA sequencing. The cDNA corresponding to the HIRMAb HC orf minus the signal peptide was generated by PCR using the pCD-HIRMAb-HC plasmid as template, and the forward ODN (SEQ ID NO:8), and the reverse ODN (SEQ ID NO:9). The forward ODN was phosphorylated for directed insertion into the AfeI site of the intermediate PON1 plasmid. The reverse ODN primer was complementary to the end of the HIRMAb-HC orf, and included the stop HC codon and the HindIII site located in the multiple cloning site on the 3′-flanking region of the HIRMAb-HC orf. The PCR generated HIRMAb-HC orf cDNA was digested with HindIII and inserted at the AfeI-HindIII sites of the intermediate PON1-AfeI plasmid to form the pPON1-HIRMAb-HC expression vector. The construct was validated by DNA sequencing, which confirmed the presence of the CMV promoter at the 5′-end of the expression cassette, and the BGH polyA sequence at the 3′-end of the expression cassette. The nucleotide sequence of the PON1-HC fusion gene is given in SEQ ID NO:24, and the deduced amino acid sequence is given in SEQ ID NO:25. The plasmid encodes for an 800 amino acid protein, comprised of the 355 amino acid PON1, which includes the amino terminal methionine, a 2 amino acid linker, and the 443 amino acid HIRMAb HC. For expression of the PON1-HIRMAb fusion protein in COS cells, the latter were co-transfected with the pPON1-HIRMAb-HC and HIRMAb-LC expression plasmid (pCD-HIRMAb-LC).

The nucleotide sequence encoding the light chain (LC) is given in SEQ ID NO:26, and the deduced amino acid sequence is given in SEQ ID NO:27. The open reading frame of the pCD-HIRMAb-LC plasmid encodes for a 234 AA light chain, which includes a 20 AA signal peptide, a 108 AA variable region of the light chain (VL), and a 106 AA human LC kappa constant region (SEQ ID NO 27).

### Example 4

### Secretion of HIRMAb-PON1 Fusion Protein by Transfected COS Cells without Lipid Acceptor

COS cells were dual transfected with the pHIRMAb-LC and either the pHIRMAb-HC-PON1 plasmid, or the pPON1-HIRMAb-HC plasmid, using Lipofectamine 2000, with a ratio of 1:2.5, ug DNA:uL Lipofectamine 2000. The pCD-HIRMAb-LC expression plasmid encodes the light chain (LC), which is used by both the engineered HIRMAb and the HIRMAb-PON1 fusion protein, or the PON1-HIRMAb fusion protein. Following transfection, the cells were cultured in serum free VP-SFM (Invitrogen, Carlsbad, Calif.), with or without 1% Ex-Cyte (Cellianca, Kankakee, Ill.), a lipoprotein supplement. The conditioned serum free medium was collected at 3 and 7 days. For screening assays, the COS cells were plated in 6-well cluster dishes. For production assays, the COS cells were plated in 10×T500 flasks with a proportionate scale-up of the Lipofectamine 2000 and the plasmid DNA.

The secretion of the HIRMAb-PON1 fusion protein into the serum free medium by transfected COS cells was monitored with an ELISA specific for human IgG. Human IgG ELISA was performed in high protein binding 96-well plates. A goat anti-human IgG primary antibody was plated in 0.1 M NaHCO3 (100 μl, 2 μg/ml) and incubated for overnight at 4 C. Plates were washed 0.01 M Na2HPO4/0.15 M NaCl/pH=7.4/0.05% Tween-20 (PBST), and blocked with 1% gelatin in PBST for 30 min at 22° C. Plates were incubated with 100 uL/well of either human IgG1 standard or the fusion protein for 60 minutes at room temperature (RT). After washing with PBST, a goat anti-human kappa LC antibody conjugated to alkaline phosphatase was plated for 60 min at 37° C. Color development was performed with p-nitrophenyl phosphate (Sigma) at pH=10.4 in the dark. The reaction was stopped with NaOH, and absorbance at 405 nm was measured in a BioRad ELISA plate reader. Human IgG1/kappa was used as the assay standard. The concentration of the HIRMAb-PON1 fusion protein, with the HC-PON1 fusion heavy chain, was 22-31 ng/mL in the absence of ExCyte, the lipid acceptor, in the medium. The concentration of the HIRMAb-PON1 fusion protein, with the PON1-HC fusion heavy chain, was 35 ng/mL in the absence of ExCyte, the lipid acceptor, in the medium. Therefore both the PON1-HIRMAb fusion protein, and the HIRMAb-PON1 fusion protein, were secreted at comparable levels, irrespective of whether the PON1 was placed at the amino terminal end of the fusion protein without a signal peptide, or was placed at the carboxyl terminal end of the fusion protein, with a leader peptide at the amino terminus.

The concentration of the HIRMAb-PON1 fusion protein, with the HC-PON1 fusion heavy chain, was 12-17 ng/mL in the presence of 1% ExCyte, the lipid acceptor, in the medium. The concentration of the PON1-HIRMAb fusion protein, with the PON1-HC fusion heavy chain, was 32 ng/mL in the presence of 1% ExCyte, the lipid acceptor, in the medium. Therefore, the presence of ExCyte in the medium had no stimulatory effect on the secretion of the secretion of either the HIRMAb-PON1 fusion protein, or the PON1-HIRMAb fusion protein, whereas ExCyte in the medium increased PON1 secretion 19-fold (Table 2). These results show fusion of PON1 to an IgG molecule allows for PON1 secretion by transfected cells without the requirement for a lipid acceptor in the medium. In contrast, transfected cells do not secrete PON1 in the absence of a lipid acceptor in the medium (Deakin et al., (2002), J Biol Chem, 277:4301-4308).

### Example 5

### PON1 Enzyme Activity of Fusion Protein Differs Widely Depending on Structure of the Fusion Protein

PON1 enzyme activity in either fusion protein was measured following affinity purification of the fusion protein. For purification of the fusion protein, COS cells were grown on 10×T500 flasks, and co-lipofected with the HC and LC expression plasmids. For production of the HIRMAb-PON1 fusion protein, the COS cells were co-lipofected with the pCD-HIRMAb-PON1 and pCD-HIRMAb-LC plasmids. For production of the PON1-HIRMAb fusion protein, the COS cells were co-lipofected with the pCD-PON1-HIRMAb and pCD-HIRMAb-LC plasmids. Following lipofection, the COS cells were cultured for up to 7 days in serum free medium without lipid acceptor. Approximately 2 liter of serum free conditioned medium was collected, and this medium was reduced to a 400 mL volume with tangential flow filtration, and the HIRMAb-PON1 fusion protein was purified by affinity chromatography with a 5 mL column of protein A Sepharose 4 Fast Flow. The protein A column was equilibrated with neutral saline buffer containing 1 mM CaCl2, and the fusion protein was eluted with 0.1 M sodium acetate/pH=3.7/1 mM CaCl2, followed by neutralization with 1 M Tris base. The neutralized acid eluate was concentrated and buffer exchanged with 0.01 M Tris/0.15 M NaCl/pH=7.4/1 mM CaCl2 with a micro-concentrator, and stored at −20 C. The protein content was measured with the bicinchoninic acid assay.

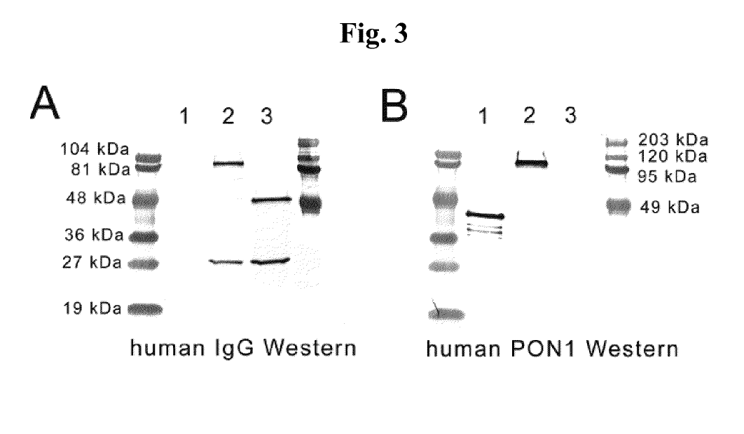

The HIRMAb-PON1 fusion protein is correctly processed as heavy and light chains following transfection of COS cells, as demonstrated by Western blotting of the affinity purified HIRMAb-PON1 fusion protein. The immunoreactivity of the HIRMAb-PON1 fusion protein was measured for both the human IgG part and the human PON1 part of the molecule. For human IgG Western blotting, the primary antibody was a goat anti-human IgG (H+L) antiserum, and binding was detected with a biotinylated horse anti-goat IgG. For human PON1 Western blotting, the primary antibody was a mouse monoclonal antibody against human PON1, and binding was detected with a biotinylated horse anti-mouse IgG. Human IgG1 and human PON1 standards were analyzed in parallel with the HIRMAb-PON1 fusion protein. On Western blotting, the LC of either the HIRMAb or the HIRMAb-PON1 fusion protein react equally with a primary antibody directed against the human IgG (H+L), as shown in FIG. 3A. The size of the HC of the fusion protein is about 40 kDa larger than the size of the HC of the HIRMAb on both Western blots using either the anti-human IgG primary antibody (FIG. 3A) or the anti-human PON1 primary antibody (FIG. 3B). The anti-PON1 primary antibody reacts with the HC of the fusion protein, and with recombinant PON1, but does not react with the HIRMAb (FIG. 3B). In the study shown in FIG. 3, the HIRMAb-PON1 fusion protein incorporated the Arg-192 allozyme. The immunoreactivity of the HIRMAb-PON1 fusion protein with the Gln-192 allozyme was also measured with the Western blot, and the results were identical to that shown in FIG. 3. Therefore, different amino acids at position 192 of the PON1 part of the fusion protein had no effect on the immunoreactivity of the fusion protein with antibodies to either human IgG1 or to PON1.

The PON1 enzyme assay was sensitive to 3 pmol/tube of enzyme reaction substrate. In the case of the PON1-HIRMAb fusion protein, where the PON1 was fused to the amino terminus of the HIRMAb heavy chain, there was no measurable PON1 enzyme activity in the fusion protein bearing the PON1-HC fusion construct, even when PON1 enzyme activity of the fusion protein was measured without dilution of the purified protein. This result indicates PON1 functional activity was completely lost when the PON1 enzyme was fused to the amino terminus of the HIRMAb heavy chain.

Next, the PON1 enzyme activity was measured in the HIRMAb-PON1 fusion protein, where the PON1 was fused to the carboxyl terminus of the HIRMAb HC, as depicted in FIG. 1. The PON1 enzyme activity of the protein A purified HIRMAb-PON1-Leu-55 fusion protein is shown in Table 3, in comparison with the PON1 enzyme activity in 20% human plasma. PON1 activity for the fusion protein is linear with respect to incubation time and concentration (Table 3). The PON1 enzyme activity of the HIRMAb-PON1/Leu-55 fusion protein, at a concentration of 68 μg/mL, is comparable to the PON1 enzyme activity in 20% human plasma (Table 3). Human plasma was used as a positive control in the PON1 fluorometric assay. The PON1 enzyme activity of 20% human plasma was 63.5±1.4 nmol/hr/mL (Table 3). Since the concentration of PON1 in human plasma is about 50 ug/mL (Paragh et al., (2006), Br J Clin Pharmacol, 61:694-701) the human plasma PON1 specific activity against the DEPFMU substrate is 6.3 nmol/hr/μg enzyme, as determined with the fluorometric assay. The PON1 specific activity against paraoxon in human plasma is 114 nmol/hr/ug enzyme, as determined with the spectrophotometric assay (Paragh supra). Therefore, the PON1 activity against DEPFMU is lower as compared to paraoxon, and this observation parallels other reports showing a reduced catalytic rate constant for DEPFMU, as compared to paraoxon, for organophosphorus hydrolase (Soukharev and Hammond, supra). The PON1 enzyme activity of the HIRMAb-PON1/Leu-55/Arg-192 fusion protein is 165±4 nmol/hr/mg (Table 3). Therefore, the PON1 enzyme specific activity of the HIRMAb-PON1 fusion protein is 0.2 nmol/hr/ug protein. Since the molecular weight of the HIRMAb-PON1 fusion protein, about 240 kDa, is about 6-fold greater than the PON1, about 40 kDa, the PON1 enzyme specific activity of the HIRMAb-PON1 fusion protein is about 20% of the native PON1 in human plasma. Since the organophosphatase enzyme activity of PON1 is >100-fold greater than any other human esterase (Josse et al., (2001), J Appl Toxicol, 21 Suppl 1:S7-11), the PON1 enzyme activity of the HIRMAb-PON1 fusion protein is a highly active organophosphatase.

### Example 6

### PON1 Enzyme Activity of Arg-192 and Gln-192 HIRMAb-PON1 Allozymes

The PON1 enzyme activity against substrates such as paraoxon is higher for the Arg-192 allozyme, as compared to the Gln-192 allozyme, although enzyme activity against chemical nerve gas agents is higher for the Gln-192 allozyme (Lenz et al, (2007), Toxicology, 233:31-39). Therefore, SDM was used to convert the Arg-192 residue to the Gln-192 in the HIRMAb-PON1 fusion protein, and the plasmid expressing the fusion heavy chain is designated the pHIRMAb-PON1-Leu55/Gln-192. The R192Q SDM was performed with a forward ODNs (SEQ ID NO:14) and a reverse ODN (SEQ ID NO:15); these ODNs were designed to introduced the mutation of interest, i.e. “A” for “G” at position 1967 of pCD-HIRMAb-PON1-Leu55 (where position 1 is the beginning of the HIRMAb-PON1 orf), and contain 15 nucleotides at each flanking region to anneal with the target sequence. The SDM was verified by bi-directional DNA sequencing. The engineering of this plasmid was validated by DNA sequencing in both directions. The nucleotide sequence of the cloned human PON1 is given in SEQ ID NO:28, and the deduced amino acid sequence of the cloned human PON1 is given in SEQ ID NO 29. COS cells were plated in 10×T500 plates and co-lipofected with the pHIRMAb-LC plasmid and either the pHIRMAb-PON1-Leu55/Arg192 plasmid, or the pHIRMAb-PON1-Leu55/Gln-192. The HIRMAb-PON1 fusion protein was purified with protein an affinity chromatography for PON1 enzyme measurements. Similar to paraoxon, the HIRMAb-PON1-Leu-55/Arg-192 allozyme has a higher enzyme activity against DEPFMU, the substrate used for the fluorometric assay, as compared to the HIRMAb-PON1-Leu-55/Gln-192 allozyme (Table 4). The PON1/Arg-192 allozyme has a higher activity toward substrates such as paraoxon as compared to the PON1/Gln-192 allozyme (Lenz et al. supra). Similarly, the HIRMAb-PON1/Arg-192 allozyme has a higher activity toward DEPFMU as compared to the HIRMAb-PON1/Gln-192 allozyme (Table 4). However, the Gln-192 allozyme may be the more powerful therapeutic form of the fusion protein. The Gln-192 polymorphism confers on PON1 both a higher enzyme activity against chemical nerve gas agents (Lenz et al., supra), and a higher anti-atherogenic effect of the enzyme (Durrington et al., (2001), Arterioscler Thromb Vasc Biol, 21:473-480).

### Example 7

### Binding of HIRMAb-PON1 Fusion Protein to the Human Insulin Receptor

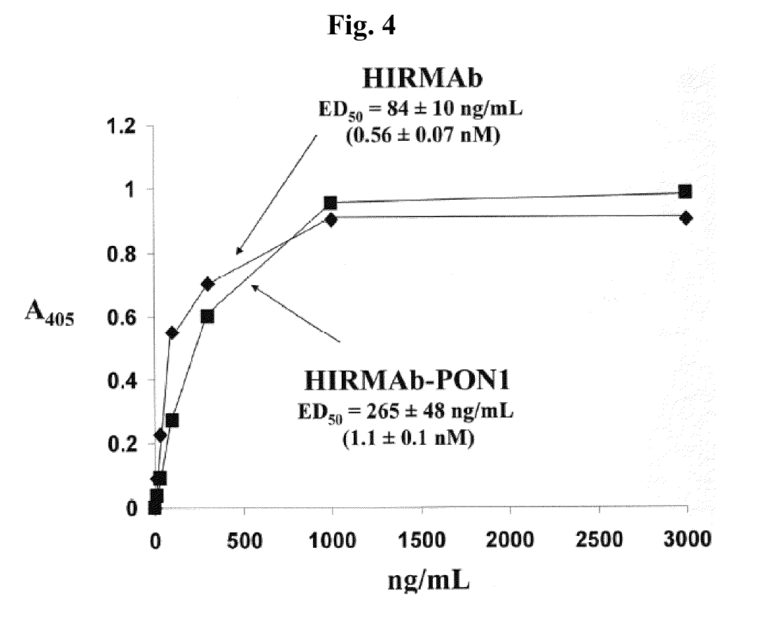

The affinity of the HIRMAb-PON1 fusion protein for the HIR extracellular domain (ECD) was determined with an ELISA using the lectin affinity purified HIR ECD. Chinese hamster ovary (CHO) cells permanently transfected with the HIR ECD were grown in serum free media, and the HIR ECD was purified with a wheat germ agglutinin affinity column. The HIR ECD (0.2 ug/well) was plated on high binding 96-well plates, and the binding of the HIRMAb, or the HIRMAb-PON1 fusion protein to the HIR ECD was detected with a biotinylated goat anti-human IgG (H+L) antibody (0.3 μg/well), and the avidin-biotinylated peroxidase detection system. The concentration that caused 50% binding to the HIR ECD, the ED50, was determined by non-linear regression analysis. There is comparable binding of either the chimeric HIRMAb or the HIRMAb-PON1 fusion protein for the HIR ECD with ED50 of 0.55±0.07 nM and 1.1±0.1 nM, respectively (FIG. 4). In the study shown in FIG. 4, the HIRMAb-PON1 fusion protein incorporated the Arg-192 allozyme. The affinity of the HIRMAb-PON1 fusion protein with the Gln-192 allozyme was also measured. In this assay, the ED50 of binding of the chimeric HIRMAb, and the HIRMAb-PON1 fusion protein, was 0.48±0.12 and 0.96±0.26 nM, respectively. Therefore, different amino acids at position 192 of the PON1 part of the fusion protein had no effect on fusion protein binding to the HIR.

### Example 8

### Genetic Engineering of Tandem Vector Encoding the HIRMAb-PON1 Fusion Protein

The HIRMAb-PON1 fusion protein is comprised of 2 heavy chains (HC) and 2 light chains (LC), as shown in FIG. 1. Therefore, the host cell must be permanently transfected with both the HC and LC genes. In addition, the host cell must be permanently transfected with a gene that allows for isolation of cell lines with amplification around the transgene insertion site. This is accomplished with selection of cell lines with methotrexate (MTX) following transfection of the host cell with a gene encoding for dihydrofolate reductase (DHFR). Therefore, it is necessary to obtain high production of all 3 genes in a single cell that ultimately produces the Master Cell Bank for manufacturing. In order to insure high expression of all 3 genes, a single piece of DNA, called a tandem vector (TV), was engineered as outlined in FIG. 5. The genetic engineering of the TV for HIRMAb-PON1 fusion protein was completed by successive insertions of both the HIRMAb LC and the DHFR genes into either the pCD-HIRMAb-PON1-R192 or -Q192 expression vectors, respectively (FIG. 5). The HIRMAb LC expression cassette comprised of the CMV promoter, the HIRMAb LC orf and the BGH pA sequence was released from the pCD-HIRMAb-LC expression vector with NruI and AfeI restriction endonuclease digestion and inserted with T4 DNA ligase at the AfeI site of the pCD-HIRMAb-PON1-R192 or -Q192, located on the 3′-flanking region of the respective BGH pA sequence (FIG. 5), to form the intermediate vectors designated pCD-HIRMAb-PON1 (R or Q192)-LC (FIG. 5). The engineering of the HIRMAb-PON1-R192 or Q192 TV was later completed by insertion of the DHFR cassette at AfeI site located on the 3′-flanking region of BGH pA region of the LC gene in the intermediate pCD-HIRMAb-PON1 (R/Q192)-LC vectors (FIG. 5). A mouse wild type (wt) DHFR expression cassette driven by the SV40 promoter and containing the hepatitis B virus transcription terminator was obtained from the pwtDFHR vector (FIG. 5) by digestion with SmaI and SalI. The SalI end was filled with T4 DNA polymerase and deoxynucleotide triphosphates prior to ligation. All intermediate vectors were treated with alkaline phosphatase to prevent self ligation.

The HIRMAb-PON1 TVs were subjected to bi-directional DNA sequencing. For the HIRMAb-PON1-R192 TV, the expression cassettes encoding the HC gene, the LC gene, and the DHFR gene, in a 5′-3′ direction were contained within 7,039 nt (SEQ ID NO:30). The HC cassette was comprised of 3513 nt, which included a 740 nt CMV promoter, a 9 nt full Kozak sequence (GCCGCCACC), the 2460 nt HC orf, and the 304 nt BGH pA sequence. The LC and HC cassettes were separated by a 23 nt linker. The LC cassette was comprised of 1736 nt, which included a 731 nt CMV promoter, a 9 nt full Kozak sequence (GCCGCCACC), the 705 nt LC orf, and the 291 nt BGH pA sequence. The DHFR cassette was comprised of 1767 nt, which included a 254 nt SV40 promoter, a 9 nt full Kozak sequence (GCCGCCACC), the 564 nt DHFR orf, and the 940 nt hepatitis B virus (HBV) pA sequence. The HC orf encoded for an 819 AA HIRMAb HC-PON1 fusion protein, which included a 19 AA signal peptide (SEQ ID NO:31). The LC orf encoded for a 234 amino acid (AA) chimeric HIRMAb LC, which included a 20 AA signal peptide (SEQ ID NO:32). The DHFR orf encoded for a 187 AA murine DHFR (SEQ ID NO:33). The fusion HC orf, the LC orf, and the DHFR orf are encoded by nt 750-3209, 4277-4981, and 5536-6099, respectively of SEQ ID NO:30.

For engineering of the tandem vector producing the HIRMAb-PON1-Q192 fusion protein, the expression cassettes encoding the HC gene, the LC gene, and the DHFR gene, in a 5′-3′ direction were contained within 7,039 nt (SEQ ID NO:34). The HC cassette was comprised of 3513 nt, which included a 740 nt CMV promoter, a 9 nt full Kozak sequence (GCCGCCACC), the 2460 nt HC orf, and the 304 nt BGH pA sequence. The LC and HC cassettes were separated by a 23 nt linker. The LC cassette was comprised of 1736 nt, which included a 731 nt CMV promoter, a 9 nt full Kozak sequence (GCCGCCACC), the 705 nt LC orf, and the 291 nt BGH pA sequence. The DHFR cassette was comprised of 1767 nt, which included a 254 nt SV40 promoter, a 9 nt full Kozak sequence (GCCGCCACC), the 564 nt DHFR orf, and the 940 nt hepatitis B virus (HBV) pA sequence. The HC orf encoded for a 819 AA HIRMAb HC-PON1 fusion protein, which included a 19 AA signal peptide (SEQ ID NO:35). The LC orf encoded for a 234 amino acid (AA) chimeric HIRMAb LC, which included a 20 AA signal peptide (SEQ ID NO:36). The DHFR orf encoded for a 187 AA murine DHFR (SEQ ID NO:37). The fusion HC orf, the LC orf, and the DHFR orf are encoded by nt 750-3209, 4277-4981, and 5536-6099, respectively of SEQ ID NO:34.

### Example 9

### Stable Transfection of CHO Cells, Dilutional Cloning, and Manufacturing

Serum free medium (SFM) adapted DG44 Chinese hamster ovary (CHO) cells (Invitrogen) were electroporated with 5 ug of the pHIRMAb-PON1 TV, following linearization with PvuI, using a Gene Pulser Xcell electroporator (BioRad, Hercules, Calif.). Five×106 cells were electroporated with the DNA in 200 uL of phosphate buffered saline (PBS) and 0.2 cm cuvettes using a square wave and 160 volts. Cells were suspended in CHO serum free medium (SFM) (Hyclone, Logan, Utah) and plated in 4×96-well plates. Selection of stable transfectants began 2 days following electroporation with 0.54 mg/ml G418. Aliquots of supernatant were taken for human IgG ELISA when colonies of transfectants were evident, i.e. 21 days. Positive clones were isolated and cultured individually for further characterization. DG44 cells lack endogenous DHFR, and rely on nutrients, hypoxanthine and thymidine (HT) for endogenous folate synthesis. Transfected cells carrying the TV express the exogenous DHFR. Transfected cell lines were further selected by placement in HT-deficient medium. Lines with amplification around the transgene insertion site were selected by subjecting the cells to increasing concentrations of MTX, starting at 20 nM MTX. Following stabilization of the cell line at 80-160 nM MTX, high producing clones were isolated by limited dilution cloning (DC) at 1 cell per well; a total of 4000 wells were plated at each round of DC, and medium IgG was measured with a human IgG ELISA using a high volume microplate dispenser and a microplate washer (Biotek, Winooski, Vt.). The cloned cells were propagated in 125 mL plastic square bottles on an orbital shaker at a viable cell density of 1-2 million cells/mL, and produced human IgG levels of approximately 10 mg/L in serum free medium, as determined by IgG ELISA. The HIRMAb-PON1 fusion protein was manufactured in a setting that could be replicated in future Good Manufacturing Practice (GMP) production for clinical trials. A 50 L Wave bioreactor was seeded with the transfected CHO cells, and the medium was expanded to 22 L. The bioreactor was maintained for approximately 3 weeks in perfusion mode, where approximately 20 L of SFM was perfused and collected each day. The viable cell density peaked at 20 million cells/mL. Approximately 230 L of conditioned medium was clarified with depth filtration, and the fusion protein was initially purified with a 1.0 L column of MAb Select Xtra in a 100/500 glass column. The protein A purified fusion protein was then purified with anion exchange chromatography and final diafiltration against 0.02 M sodium Tris/0.15 M NaCl/pH=8.0/1 mM CaCl2/0.025% Tergitol NP-10 buffer. CHO host protein was 4 parts per million (PPM), as determined by ELISA (Cygnus Technologies, Southport, N.C.); protein A was 21 PPM, as determined by ELISA (Cygnus Technologies); DNA was <0.01 PPM as determined by real time PCR using CHO cell DNA as the assay standard; and endotoxin was <0.1 EU/mg protein, as determined by the limulus amebocyte lysate assay (Lonza Biologics, Portsmouth, N.H.). The final product was a clear, colorless solution of 2.3 mg/mL, and conformed to specifications with regard to identity (human IgG and PON1 Western blotting), potency (HIR binding affinity, PON1 enzyme activity), and purity [sodium dodecylsulfate polyacrylamide gel electrophoresis (SDS-PAGE), size exclusion high performance liquid chromatography]. The protein was stable as a sterile liquid stored at 4 C for at least several months, with no change in binding affinity for the HIR or change in PON1 enzyme activity, as compared to the Reference Standard. The PON1 enzyme specific activity of the HIRMAb-PON1 fusion protein was 599±23 nmol/hr/mg protein, as determined with a fluorometric enzymatic assay. This enzyme activity was normalized for the molecular weight of the fusion protein, which yielded a PON1 enzyme specific activity of 67±3 nmol/hr/mmol of fusion protein. The PON1 enzyme activity of human plasma was 68±1 nmol/hr/mmol PON1, assuming a PON1 molecular weight of 39,773 Da. Therefore, the PON1 enzyme activity of the HIRMAb-PON1 fusion protein produced by and purified from CHO cells was identical to the enzyme activity of PON1 in human plasma.

### Example 10

### Variation of Human Constant Regions

The domain structure of the HC of the fusion protein, including the complementarity determining regions (CDRs) and framework regions (FR) of the chimeric HIRMAb HC are given in FIG. 6. The constant region is derived from human IgG1, and the amino acid sequence comprising the CH1, hinge, CH2, and CH3 is given in FIG. 6. The domain structure of the LC, including the CDRs and FRs of the chimeric HIRMAb LC are given in FIG. 7. The constant region is derived from human kappa LC, and the amino acid sequence comprising the human kappa constant region is given in FIG. 7.

The constant (C)-region of the HIRMAb HC-PON1 fusion protein listed in SEQ ID NOs:21, 23, and 25 is derived from the human IgG1 isotype. In addition, the HC C-region could be derived from the C-region of other human IgG isotypes, including human IgG2, IgG3, and IgG4. The different C-region isotypes each offer well known advantages or disadvantages pertaining to flexibility around the hinge region, protease sensitivity, activation of complement or binding to the Fc receptor. The C-region of the HIRMAb LC is given in SEQ ID NO:27, and is from the human kappa isotype. In addition, the light chain C-region could be derived from the human lambda light chain isotype.

### Example 11

### Secretion of PON1 by Transfected Host Cells as IgG Fc Fusion Protein

In addition to fusion to the HIRMAb, the recombinant human PON1 could be fused to a human Fc fragment derived from the C-region of different human IgG isotypes, for the purposes of enhancing secretion from transfected host cells, in the absence of a lipid acceptor. The PON1 could be fused to either the amino terminus or the carboxyl terminus of the Fc fragment of human IgG1. In one embodiment, the Fc fragment includes the hinge, CH2 and CH3 region shown in FIG. 6. The amino acid sequence of the human IgG1 hinge, CH2, and CH3 are given in positions 231-242, 243-355, 356-462, respectively of SEQ ID NO:21. Alternatively, the human IgG1 Fc region could be substituted with the Fc region from other isotypes, including human IgG2, IgG3, or IgG4. If the PON1 is fused to the carboxyl terminus of the Fc region, then an IgG signal peptide, or other eukaryotic signal peptide, can be fused to the amino terminus of the Fc region to enhance secretion of the Fc-PON1 fusion protein. The production of a Fc-PON1 fusion protein provides an alternative to the production of recombinant PON1 by host cells. PON1 is not secreted in the absence of a lipid acceptor (Deakin et al., supra). The requirement of a lipid acceptor is problematic in the manufacturing of PON1 as a therapeutic product, because harsh detergents are required to separate PON1 from the lipid acceptor (Josse et al., (2002) J. Biol. Chem., 277:33386-33397).

### Example 12

### Variation of the Linker Separating the IgG Chain and the PON1

The heavy chain-PON1 fusion proteins described above were engineered with a linker comprised of either 2 amino acids (Ser-Ser), or 3 amino acids (Ser-Ser-Ser) between the IgG heavy chain and the PON1. In the sequence described in SEQ ID NO:21, there is a Ser-Ser linker at amino acids 463-464. In the sequence described in SEQ ID NO:25, there is a Ser-Ser linker at amino acids 463-464. In the sequences described in SEQ ID NOs:23, 29, 31, and 35, there is a Ser-Ser-Ser linker at amino acids 462-464.

A variety of other linkers could be used to join the IgG chain and the PON1. For example, a pCD-HIRMAb-PON1 HC expression plasmid was engineered, which produces an extended linker between the HIRMAb HC and the PON1. The HIRMAb-PON1-Leu55 fusion protein HC was used as template, and a GGGGSGGGGSGGGGS linker, designated GS15 (SEQ ID NO:38), was introduced at the original SS linker to form the extended linker SGGGGSGGGGSGGGGSS (SEQ ID NO:39). The engineering of the pCD-HIRMAb-PON1-GS15 expression plasmid was performed in 2 steps. First, an AfeI was introduced by SDM at the “SS” linker between the CH3 of the HIRMAb HC and PON1 orf to allow for insertion of the GS15 linker. The SDM was completed with a forward ODN (SEQ ID NO:10) and a reverse ODN (SEQ ID NO:11). ODNs were designed to introduced the mutation of interest and contain 15 nucleotides at each flanking region to anneal with the target sequence. The SDM was verified by bi-directional DNA sequencing. Secondly, the GS15 linker was inserted at the AfeI site to form the pCD-HIRMAb-PON1-GS15 expression plasmid. The double stranded GS15 cDNA was prepared with complementary ODNs (SEQ ID NO:12 and 13), which were phosphorylated for direct insertion into the AfeI site of the intermediate vector. The DNA sequence of the pCD-HIRMAb-PON1-GS15 vector, and the expected amino acid sequence, was verified by bi-directional DNA sequencing.

### Example 13

### Deletion of PON1 Signal Peptide

PON1 has a 15 amino acid signal peptide, which is not cleaved in vivo. Only the amino terminal methionine is cleaved. The PON1 signal peptide is an internal sequence in the heavy chain of the HIRMAb-PON1 fusion protein, since the PON1 is fused to the carboxyl terminus of the HIRMAb heavy chain. Theoretically, it might be possible for this signal peptide to undergo cleavage in vivo, which would result in a separation of the PON1 and the HIRMAb. Therefore, a modified HIRMAb-PON1 fusion protein was engineered and expressed, wherein the 15 amino acid signal peptide of PON1 was deleted from the fusion heavy chain. For the engineering of the pCD-HIRMAb-PON1-(minus signal peptide) plasmid, the human mature PON1 cDNA corresponding to amino acids Leu16-Leu355 was cloned by PCR using the pCD-HIRMAb-PON1 (Q192) as template (FIG. 5). The PCR cloning reaction was performed with forward (SEQ ID NO 40) and reverse (SEQ ID NO 2) ODN primers, respectively, as described in Example 3. The PCR ODNs are 5′-phosphorylated for direct insertion into the HpaI site of the pCD-HIRMAb-HC expression plasmid (FIG. 2B). The engineering of the gene encoding the HIRMAb-PON1-(minus signal peptide) fusion protein was performed by ligation of the ˜1.0 kb PCR generated PON1 cDNA (i.e. PON1-Leu16-Leu355) into the single HpaI site of the universal HIRMAb-HC vector (pCD-HIRMAb-HC, FIG. 2B). Following linearization of the pCD-HIRMAb-HC with HpaI, the plasmid was treated with alkaline phosphatase to prevent self-ligation. The PON1 forward PCR primer (SEQ ID NO 40) introduces “CA” nucleotides to maintain the open reading frame and to introduce a Ser-Ser linker between the carboxyl terminus of the CH3 region of the HIRMAb HC and the amino terminus of the PON1. The PON1 reverse PCR primer (Table 1, SEQ ID NO 2) introduces a stop codon, “TGA,” immediately after the terminal leucine of the mature PON1 protein. The engineered pCD-HIRMAb-PON1-Leu16-Leu355 expression vector was validated by DNA sequencing, which confirmed the presence of the CMV promoter at the 5′-end of the expression cassette, and the BGH polyA sequence at the 3′-end of the expression cassette. The deduced amino acid sequence of the HIRMAb-PON1-Leu16-Leu355 HC fusion protein is given in SEQ ID NO 41. The HIRMAb-PON1-Leu16-Leu355 plasmid encodes for a 804 amino acid protein, comprised of a 19 amino acid IgG signal peptide, a 113 amino acid variable region of the heavy chain (VH) of the HIRMAb, a 330 amino acid human IgG1 constant region, a 2 amino acid linker (Ser-Ser), and the 340 amino acid human PON1 (Leu16-Leu355). The predicted molecular weight of the HIRMAb-PON1-Met55 heavy chain fusion protein, minus glycosylation, is 86,964 Da, with a predicted pI of 6.11. The HIRMAb-PON1-Leu16-Leu355 fusion protein was validated by transient expression in COS cells following co-transfection with pCD-HIRMAb-Leu16-Leu355 and pCD-HIRMAb-LC. The levels of protein in the conditioned medium of COS cells 7 day after transfection were 30-fold increased for the HIRMAb-PON1-Leu16-Leu355 fusion protein as compared with the parental HIRMAb-PON1-Met1-Leu355 fusion protein.

### Example 14

### Treatment of Chemical Nerve Gas Organophosphate Exposure with the HIRMAb-PON1 Fusion Protein

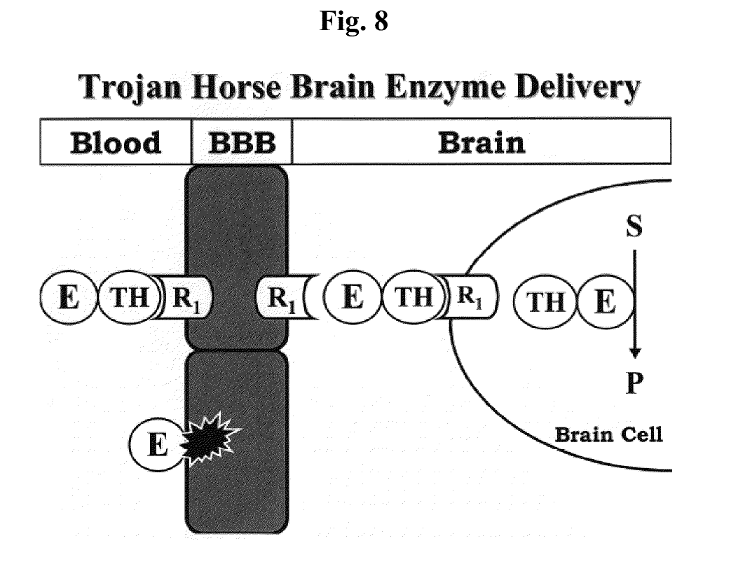

Chemical nerve gas agents rapidly cross the BBB, enter brain, inhibit cerebral ACE, and cause death. Consequently, an organophosphatase treatment of chemical nerve gas exposure should seek to inactivate the organophosphates (OPs) in both brain and plasma. The delivery of PON1 into brain cells is depicted in FIG. 8. An enzyme (E, FIG. 8), such as PON1, does not cross the BBB and cannot enter brain. Following fusion of the enzyme to the molecular Trojan horse (TH, FIG. 8), such as the HIRMAb, the TH-E fusion protein is able to cross the BBB via receptor-mediated transport on the BBB insulin receptor recognized by the HIRMAb (R1, FIG. 8). Once across the BBB, the HIRMAb-PON1 fusion protein may inactivate OPs in brain interstitial space. However, the neuronal cell membrane also expresses the insulin receptor, and the HIRMAb-PON1 fusion protein may undergo receptor-mediated endocytosis into neurons to inactive intracellular OPs.

The KI of ACE inhibition for chemical nerve gas agents, such as VX, soman, sarin, or tabun, ranges from 0.36 nM to 3.5 nM (Augerson, (2001) Chemical and Biological Warfare Agents, RAND Corp. Therefore, the goal in treatment of chemical nerve gas penetration in the CNS is to achieve a cerebral concentration of 1 nM PON1, which is equal to the KI of ACE inhibition by the organophosphate. Since the PON1 is fused to a single heavy chain of the HIRMAb (FIG. 1), the effective molecular weight of the active PON1 fusion protein is approximately 100,000 Da. A 1 nM concentration is therefore equivalent to a HIRMAb-PON1 concentration of 100 ng/mL of brain or approximately 0.1 ug/gram brain. Based on pharmacokinetic studies of HIRMAb-enzyme fusion proteins in the primate, the expected brain concentration in humans is 0.5% of injected dose (ID)/brain (Boado et al., (2008), Biotechnol Bioeng., 99:475-484). Since the human brain weighs approximately 1,000 grams, an injection dose of the HIRMAb-PON1 fusion protein of 20 mg is projected to generate a brain concentration of 0.1 ug/g. With formulation of the HIRMAb-PON1 fusion protein at a concentration of 20 mg/mL, the effective treatment volume of drug would be 1.0 mL, which could be administered as a rapid intra-muscular injection to rapid response personnel, in a civilian chemical nerve gas attack, or to military personnel, in a warfield chemical nerve gas attack. Alternatively, if the goal is to achieve a saturating concentration of PON1 enzyme activity in the brain, such as 1 ug/gram brain, then the dose of HIRMAb-PON1 fusion protein would be 200 mg, and this could be formulated in a 5 mg/mL solution, which is administered as a rapid intravenous infusion of 40 mL over a 10-20 min period.

In addition to inactivation of chemical nerve gas agents in the brain, the administration of the HIRMAb-PON1 fusion protein at doses of 20-200 mg would be expected to produce elevated plasma concentrations of the fusion protein. Based on pharmacokinetic studies of HIRMAb-enzyme fusion proteins in the primate (Boado et al, supra), the expected plasma concentration in humans is 0.01% of injected dose (ID)/mL, which would be equivalent to HIRMAb-PON1 fusion protein concentrations of 2 and 20 ug/mL at the doses of 20 and 200 mg, respectively. These high concentrations of fusion protein would degrade chemical nerve gas agents in the blood, prior to penetration into the brain.

### Example 15

### Treatment of Cerebral Atherosclerosis with the HIRMAb-PON1 Fusion Protein

PON1 knockout mice have an increased in vascular atherosclerosis, in addition to an increased sensitivity to organophosphates. PON1 deficient mice have an increase in vascular adhesion, oxidative stress, and thrombotic tendencies, even in the absence of hyperlipidemia (Ng et al, (2008), Cardiovasc. Pathol., in press). PON1 may reduce oxidation of vascular lipids, and thereby slow the progress of vascular atherosclerosis. The role of PON1 in reduction of vascular atherosclerosis has also been implicated in cerebral vascular disease. Decreased plasma PON1 levels are a risk factor for ischemic stroke caused by cerebral atherosclerosis (Kim et al., (2007), Biochem. Biophys. Res. Comm., 364:157-172). Vascular disease in brain is caused by changes in the smooth muscle layer in cerebral vessels. The smooth muscle cells are located distal to the endothelium in brain vessels. Since the BBB is formed by the endothelium, the smooth muscle cells of cerebral vessels are behind the BBB. Therefore, the administration of recombinant PON1 would not be effective in cerebral atherosclerosis, because a large molecule such as PON1 does not cross the BBB, as depicted in FIG. 8. In contrast, PON1 fused to the HIRMAb would cross the BBB via receptor-mediated transport and distribute to smooth muscle cells in brain vessels, which also express the insulin receptor, as depicted in FIG. 8. The distribution of exogenous PON1 to cerebral vascular smooth muscle cells may reduce the progression of cerebral atherosclerosis, and thereby reduce the incidence of stroke. There are over 800,000 cases of stroke in the U.S. alone, which is a major source of mortality and morbidity. The chronic administration of a HIRMAb-PON1 fusion protein could prove to be a new treatment for chronic cerebral atherosclerosis.

TABLE 1
Oligodeoxynucleotide primers used in the RT-PCR
cloning of human PON1

| | |
|---|---|
| PON1 FWD: 5′-phosphate-CCCGACCATGGCGAAGCTGATTG | (SEQ ID NO: 1) |
| PON1 REV: 5′-phosphate-CGGTCTGTTAGAGCTCACAGTAAAG | (SEQ ID NO: 2) |

TABLE 2
PON1 enzyme activity in the medium of COS cells following
transfection with pCD-PON1

PON1 enzyme activity
(nmol/hr/mL)

| Sample | 0% ExCyte | 1% ExCyte |
|---|---|---|
| COS medium, pCD-PON1 | 1.24 ± 0.01 | 23.3 ± 0.1 |
| COS medium, Lipofectamine 2000 | 0.26 ± 0.02 | 0.29 ± 0.01 |
| Human plasma (10%) | 41.3 ± 3.1 | n.m. |

Mean ± SE (n = 3 dishes per point);
n.m. = not measured.
Clone pCD-PON1 produces the Met-55/Arg-192 allozyme; serum free medium was harvested 7 days after transfection of COS cells with pCD-PON1.

TABLE 3
PON1 enzyme activity of protein A purified HIRMAb-PON1
fusion protein

| | | | PON1 enzyme activity |
|---|---|---|---|
| | assay units | | (nmol/hr/mL or |
| Enzyme source | 20 min | 40 min | nmol/hr/mg) |
| Human plasma (20%) | 140 ± 2 | 273 ± 1 | 63.5 ± 1.0 (S.E.) |
| HIRMAb-PON1/ | | | |
| Leu-55/Arg-192 | | | |
| 270 μg/mL | 492 ± 6 | 999 ± 10 | 165 ± 4 |
| 68 μg/mL | 117 ± 2 | 233 ± 1 | 156 ± 4 |

Fluorometric assay units were converted into enzyme activity based on the standard curve. The units of human plasma PON1 enzyme activity are nmol/hr/mL; the units of HIRMAb-PON1 enzyme activity are nmol/hr/mg protein.

TABLE 4
PON1 enzyme activity of protein A purified HIRMAb-PON1/Arg-192
and HIRMAb-PON1/Gln-192 fusion protein allozymes

| | assay units ± S.E. | | |
|---|---|---|---|
| Enzyme source | 10 min | 20 min | 40 min |
| HIRMAb-PON1/ | | | |
| Leu-55/Arg-192 | | | |
| 270 μg/mL | 184 ± 1 | 362 ± 2 | 717 ± 4 |
| 135 μg/mL | 92 ± 1 | 183 ± 2 | 363 ± 2 |
| 68 μg/mL | 45 ± 2 | 92 ± 1 | 181 ± 2 |
| HIRMAb-PON1/ | | | |
| Leu-55/Gln-192 | | | |
| 500 μg/mL | 100 ± 2 | 200 ± 1 | 404 ± 2 |
| 250 μg/mL | 52 ± 1 | 101 ± 2 | 203 ± 2 |
| 125 μg/mL | 26 ± 2 | 52 ± 1 | 101 ± 2 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed:

1. A method for treating organophosphate intoxication in a subject in need thereof, comprising systemically administering to the subject a dose of a fusion antibody having organophosphatase activity and binding to the extracellular domain of a receptor expressed on the BBB.

2. The method of claim 1, wherein the receptor is an insulin receptor, a transferrin receptor, or a lipoprotein receptor.

3. The method of claim 2, wherein the fusion antibody binds to an extracellular domain of the human insulin receptor.

4. The method of claim 1, wherein at least about 0.5% of the systemically administered dose is delivered to the brain.

5. The method of claim 1, wherein the fusion antibody comprises an amino acid sequence at least 70% identical to the amino acid sequence of human PON1.

6. The method of claim 1, wherein the subject is suffering from acute exposure to an organophosphate.

7. The method of claim 1, wherein the subject is suffering from chronic exposure to an organophosphate.

8. The method of claim 1, wherein the fusion antibody comprises an immunoglobulin heavy chain comprising a CDR1 corresponding to the amino acids 45-54 of SEQ ID NO:21 with up to 4 single amino acid mutations, a CDR2 corresponding to the amino acids 69-85 of SEQ ID NO:21 with up to 6 single amino acid mutations, or a CDR3 corresponding to the amino acids 118-121 of SEQ ID NO:21 with up to 3 single amino acid mutations, wherein the single amino acid mutations are substitutions, deletions, or insertions.

9. The method of claim 1, wherein the fusion antibody comprises an immunoglobulin heavy chain comprising a CDR1 corresponding to the amino acids 45-54 of SEQ ID NO:21 with up to 4 single amino acid mutations, a CDR2 corresponding to the amino acids 69-85 of SEQ ID NO:21 with up to 6 single amino acid mutations, or a CDR3 corresponding to the amino acids 118-121 of SEQ ID NO:21 with up to 5 single amino acid mutations, wherein the single amino acid mutations are substitutions, deletions, or insertions.

10. A method for protecting a subject from organophosphate intoxication comprising systemically administering to a subject at high risk of organophosphate intoxication a dose of a fusion antibody having organophosphatase activity and binding to the extracellular domain of a receptor expressed on the BBB.

11. The method of claim 10, wherein the receptor is an insulin receptor, a transferrin receptor, or a lipoprotein receptor.

12. The method of claim 10, wherein at least about 0.5% of the systemically administered dose is delivered to the brain.

13. The method of claim 10, wherein the fusion antibody comprises an amino acid sequence at least 70% identical to the amino acid sequence of human PON1.

14. The method of claim 10, wherein the fusion antibody binds to an extracellular domain of the human insulin receptor

15. A method for treating of cerebral atherosclerosis, comprising administering to a subject in need thereof a dose of a fusion antibody that has organophosphatase activity and binds to the extracellular domain of a receptor expressed on the BBB.

16. A fusion antibody comprising a heavy chain immunoglobulin or a light chain immunoglobulin covalently linked to an organophosphatase, wherein the fusion antibody binds to the extracellular domain of a receptor expressed on the BBB.

17. The fusion antibody of claim 16, wherein the receptor is an insulin receptor, a transferrin receptor, or a lipoprotein receptor

18. The fusion antibody of claim 16, wherein the fusion antibody binds to an extracellular domain of the human insulin receptor

19. The fusion antibody of claim 16, wherein the fusion antibody comprises an immunoglobulin heavy chain comprising a CDR1 corresponding to the amino acid s 45-54 of SEQ ID NO:21 with up to 4 single amino acid mutations, a CDR2 corresponding to the amino acids 69-85 of SEQ ID NO:21 with up to 6 single amino acid mutations, or a CDR3 corresponding to the amino acids 118-121 of SEQ ID NO:21 with up to 3 single amino acid mutations, wherein the single amino acid mutations are substitutions, deletions, or insertions.

20. The fusion antibody of claim 19, wherein the fusion antibody comprises an immunoglobulin heavy chain comprising a CDR1 corresponding to the amino acids 45-54 of SEQ ID NO:21 with up to 4 single amino acid mutations, a CDR2 corresponding to the amino acids 69-85 of SEQ ID NO:21 with up to 6 single amino acid mutations, or a CDR3 corresponding to the amino acids 118-121 of SEQ ID NO:21 with up to 5 single amino acid mutations, wherein the single amino acid mutations are substitutions, deletions, or insertions.

21. The fusion antibody of claim 16, wherein the immunoglobulin light chain comprises a CDR1 corresponding to the amino acids 45-54 of SEQ ID NO:21 with up to 4 single amino acid mutations, a CDR2 corresponding to the amino acids 69-85 of SEQ ID NO:21 with up to 6 single amino acid mutations, or a CDR3 corresponding to the amino acids 118-121 of SEQ ID NO:21 with up to 5 single amino acid mutations, wherein the single amino acid mutations are substitutions, deletions, or insertions.

22. The fusion antibody of claim 16, wherein the fusion antibody competes for binding to the human insulin receptor with an antibody comprising a heavy chain comprising amino acids 20-462 of SEQ ID NO 21.


SEQUENCE LISTING (63 sequences)

SEQ ID NO: 1 - DNA, 23 bp, Artificial Sequence
  Description of Artificial Sequence Synthetic primer
cccgaccatggcgaagctgattg

SEQ ID NO: 2 - DNA, 25 bp, Artificial Sequence
  Description of Artificial Sequence Synthetic primer
cggtctgttagagctcacagtaaag

SEQ ID NO: 3 - DNA, 31 bp, Artificial Sequence
  Description of Artificial Sequence Synthetic oligonucleotide
actggctctgaagacttggagatactgccta

SEQ ID NO: 4 - DNA, 31 bp, Artificial Sequence
  Description of Artificial Sequence Synthetic oligonucleotide
taggcagtatctccaagtcttcagagccagt

SEQ ID NO: 5 - DNA, 24 bp, Artificial Sequence
  Description of Artificial Sequence Synthetic oligonucleotide
caatggctaagctgattgcgctca

SEQ ID NO: 6 - DNA, 36 bp, Artificial Sequence
  Description of Artificial Sequence Synthetic oligonucleotide
ctttactgtgagctcagcgctaccgatctgcagaat

SEQ ID NO: 7 - DNA, 36 bp, Artificial Sequence
  Description of Artificial Sequence Synthetic oligonucleotide
attctgcagatcggtagcgctgagctcacagtaaag

SEQ ID NO: 8 - DNA, 23 bp, Artificial Sequence
  Description of Artificial Sequence Synthetic oligonucleotide
agccaggttcagctgcagcagtc

SEQ ID NO: 9 - DNA, 26 bp, Artificial Sequence
  Description of Artificial Sequence Synthetic oligonucleotide
gtttaaacttaagcttggtaccgagc

SEQ ID NO: 10 - DNA, 36 bp, Artificial Sequence
  Description of Artificial Sequence Synthetic oligonucleotide
ctgtctccgggtaaaagcgctcaatggctaagctga

SEQ ID NO: 11 - DNA, 36 bp, Artificial Sequence
  Description of Artificial Sequence Synthetic oligonucleotide
tcagcttagccattgagcgcttttacccggagacag

SEQ ID NO: 12 - DNA, 43 bp, Artificial Sequence
  Description of Artificial Sequence Synthetic oligonucleotide
ggaggcggaggttctggaggcggaggttctggaggcggaggta

SEQ ID NO: 13 - DNA, 43 bp, Artificial Sequence
  Description of Artificial Sequence Synthetic oligonucleotide
tacctccgcctccagaacctccgcctccagaacctccgcctcc

SEQ ID NO: 14 - DNA, 31 bp, Artificial Sequence
  Description of Artificial Sequence Synthetic oligonucleotide
ttgacccctacttacaatcctgggagatgta

SEQ ID NO: 15 - DNA, 31 bp, Artificial Sequence
  Description of Artificial Sequence Synthetic oligonucleotide
tacatctcccaggattgtaagtaggggtcaa

SEQ ID NO: 16 - DNA, 1068 bp, Artificial Sequence
  Description of Artificial Sequence Synthetic polynucleotide
atggcgaagctgattgcgctcaccctcttggggatgggactggcactcttcaggaaccaccagtcttcttaccaaacacgacttaatgctctccgagaggtacaacccgtagaacttcctaactgtaatttagttaaaggaatcgaaactggctctgaagacatggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttacgatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaa

SEQ ID NO: 17 - protein, 355 aa, Homo sapiens [sequence omitted]

SEQ ID NO: 18 - DNA, 1068 bp, Artificial Sequence
  Description of Artificial Sequence Synthetic polynucleotide
atggcgaagctgattgcgctcaccctcttggggatgggactggcactcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttacgatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaa

SEQ ID NO: 19 - protein, 355 aa, Homo sapiens [sequence omitted]

SEQ ID NO: 20 - DNA, 2460 bp, Artificial Sequence
  Description of Artificial Sequence Synthetic polynucleotide
atggactggacctggagggtgttctgcctgcttgcagtggcccccggagcccacagccaggttcagctgcagcagtctggacctgagctggtgaagcctggggctttagtgaagatatcctgcaaggcttctggttacaccttcacaaactacgatatacactgggtgaagcagaggcctggacagggacttgagtggattggatggatttatcctggagatggtagtactaagtacaatgagaaattcaagggcaaggccacactgactgcagacaaatcctccagcacagcctacatgcacctcagcagcctgacttctgagaaatctgcagtctatttctgtgcaagagagtgggcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaagttcaatggctaagctgattgcgctcaccctcttggggatgggactggcactcttcaggaaccaccagtcttcttaccaaacacgacttaatgctctccgagaggtacaacccgtagaacttcctaactgtaatttagttaaaggaatcgaaactggctctgaagacatggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttacgatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaa

SEQ ID NO: 21 - protein, 819 aa, Artificial Sequence
  Description of Artificial Sequence Synthetic polypeptide
MDWTWRVFCLLAVAPGAHSQVQLQQSGPELVKPGALVKISCKASGYTFTNYDIHWVKQRPGQGLEWIGWIYPGDGSTKYNEKFKGKATLTADKSSSTAYMHLSSLTSEKSAVYFCAREWAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSSMAKLIALTLLGMGLALFRNHQSSYQTRLNALREVQPVELPNCNLVKGIETGSEDMEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLRSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 22 - DNA, 2460 bp, Artificial Sequence
  Description of Artificial Sequence Synthetic polynucleotide
atggactggacctggagggtgttctgcctgcttgcagtggcccccggagcccacagccaggttcagctgcagcagtctggacctgagctggtgaagcctggggctttagtgaagatatcctgcaaggcttctggttacaccttcacaaactacgatatacactgggtgaagcagaggcctggacagggacttgagtggattggatggatttatcctggagatggtagtactaagtacaatgagaaattcaagggcaaggccacactgactgcagacaaatcctccagcacagcctacatgcacctcagcagcctgacttctgagaaatctgcagtctatttctgtgcaagagagtgggcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcctggtagtagttcaatggctaagctgattgcgctcaccctcttggggatgggactggcactcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttacgatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaa

SEQ ID NO: 23 - protein, 819 aa, Artificial Sequence
  Description of Artificial Sequence Synthetic polypeptide
MDWTWRVFCLLAVAPGAHSQVQLQQSGPELVKPGALVKISCKASGYTFTNYDIHWVKQRPGQGLEWIGWIYPGDGSTKYNEKFKGKATLTADKSSSTAYMHLSSLTSEKSAVYFCAREWAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSSSMAKLIALTLLGMGLALFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLRSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 24 - DNA, 2403 bp, Artificial Sequence
  Description of Artificial Sequence Synthetic polynucleotide
atggcgaagctgattgcgctcaccctcttggggatgggactggcactcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttacgatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctcagcagccaggttcagctgcagcagtctggacctgagctggtgaagcctggggctttagtgaagatatcctgcaaggcttctggttacaccttcacaaactacgatatacactgggtgaagcagaggcctggacagggacttgagtggattggatggatttatcctggagatggtagtactaagtacaatgagaaattcaagggcaaggccacactgactgcagacaaatcctccagcacagcctacatgcacctcagcagcctgacttctgagaaatctgcagtctatttctgtgcaagagagtgggcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 25 - protein, 800 aa, Artificial Sequence
  Description of Artificial Sequence Synthetic polypeptide
MAKLIALTLLGMGLALFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLRSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCELSSQVQLQQSGPELVKPGALVKISCKASGYTFTNYDIHWVKQRPGQGLEWIGWIYPGDGSTKYNEKFKGKATLTADKSSSTAYMHLSSLTSEKSAVYFCAREWAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - DNA, 705 bp, Artificial Sequence
  Description of Artificial Sequence Synthetic polynucleotide
atggagacccccgcccagctgctgttcctgttgctgctttggcttccagatactaccggcgacatccagatgacccagtctccatcctccttatctgcctctctgggagaaagagtcagtctcacttgtcgggcaagtcaggacattggtggtaacttatactggcttcagcagggaccagatggaactattaaacgcctgatctacgccacatccagtttagattctggtgtccccaaaaggttcagtggcagtaggtctgggtcagattattctctcaccatcagcagccttgagtctgaagattttgtagactattactgtctacagtattctagttctccgtggacgttcggtggaggcacaaagatggaaataaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 27 - protein, 234 aa, Artificial Sequence
  Description of Artificial Sequence Synthetic polypeptide
METPAQLLFLLLLWLPDTTGDIQMTQSPSSLSASLGERVSLTCRASQDIGGNLYWLQQGPDGTIKRLIYATSSLDSGVPKRFSGSRSGSDYSLTISSLESEDFVDYYCLQYSSSPWTFGGGTKMEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - DNA, 2460 bp, Artificial Sequence
  Description of Artificial Sequence Synthetic polynucleotide
atggactggacctggagggtgttctgcctgcttgcagtggcccccggagcccacagccaggttcagctgcagcagtctggacctgagctggtgaagcctggggctttagtgaagatatcctgcaaggcttctggttacaccttcacaaactacgatatacactgggtgaagcagaggcctggacagggacttgagtggattggatggatttatcctggagatggtagtactaagtacaatgagaaattcaagggcaaggccacactgactgcagacaaatcctccagcacagcctacatgcacctcagcagcctgacttctgagaaatctgcagtctatttctgtgcaagagagtgggcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgaaaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcctggtagtagttcaatggctaagctgattgcgctcaccctcttggggatgggactggcactcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttacaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaa

SEQ ID NO: 29 - protein, 819 aa, Artificial Sequence
  Description of Artificial Sequence Synthetic polypeptide
MDWTWRVFCLLAVAPGAHSQVQLQQSGPELVKPGALVKISCKASGYTFTNYDIHWVKQRPGQGLEWIGWIYPGDGSTKYNEKFKGKATLTADKSSSTAYMHLSSLTSEKSAVYFCAREWAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSSSMAKLIALTLLGMGLALFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLQSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 30 - DNA, 7039 bp, Artificial Sequence
  Description of Artificial Sequence Synthetic polynucleotide
tcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggagccgccaccatggactggacctggagggtgttctgcctgcttgcagtggcccccggagcccacagccaggttcagctgcagcagtctggacctgagctggtgaagcctggggctttagtgaagatatcctgcaaggcttctggttacaccttcacaaactacgatatacactgggtgaagcagaggcctggacagggacttgagtggattggatggatttatcctggagatggtagtactaagtacaatgagaaattcaagggcaaggccacactgactgcagacaaatcctccagcacagcctacatgcacctcagcagcctgacttctgagaaatctgcagtctatttctgtgcaagagagtgggcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcctggtagtagttcaatggctaagctgattgcgctcaccctcttggggatgggactggcactcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttacgatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaacagaccgaacccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcaggccgccaccatggagacccccgcccagctgctgttcctgttgctgctttggcttccagatactaccggcgacatccagatgacccagtctccatcctccttatctgcctctctgggagaaagagtcagtctcacttgtcgggcaagtcaggacattggtggtaacttatactggcttcagcagggaccagatggaactattaaacgcctgatctacgccacatccagtttagattctggtgtccccaaaaggttcagtggcagtaggtctgggtcagattattctctcaccatcagcagccttgagtctgaagattttgtagactattactgtctacagtattctagttctccgtggacgttcggtggaggcacaaagatggaaataaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagcgggaggtaccgagctcttacgcgtgctagctcgagatatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttatcgattctagaagccgccaccatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatccttcgcgggacgtcctttgtttacgtcccgtcggcgctgaatcccgcggacgacccctcgcggggccgcttgggactctctcgtccccttctccgtctgccgttccagccgaccacggggcgcacctctctttacgcggtctccccgtctgtgccttctcatctgccggtccgtgtgcacttcgcttcacctctgcacgttgcatggagaccaccgtgaacgcccatcagatcctgcccaaggtcttacataagaggactcttggactcccagcaatgtcaacgaccgaccttgaggcctacttcaaagactgtgtgtttaaggactgggaggagctgggggaggagattaggttaaaggtctttgtattaggaggctgtaggcataaattggtctgcgcaccagcaccatgcaactttttcacctctgcctaatcatctcttgtacatgtcccactgttcaagcctccaagctgtgccttgggtggctttggggcatggacattgacccttataaagaatttggagctactgtggagttactctcgtttttgccttctgacttctttccttccgtcagagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcg

SEQ ID NO: 31 - protein, 819 aa, Artificial Sequence; identical to SEQ ID NO: 23
  Description of Artificial Sequence Synthetic polypeptide

SEQ ID NO: 32 - protein, 234 aa, Artificial Sequence; identical to SEQ ID NO: 27
  Description of Artificial Sequence Synthetic polypeptide

SEQ ID NO: 33 - protein, 187 aa, Mus sp.
MVRPLNCIVAVSQNMGIGKNGDLPWPPLRNEFKYFQRMTTTSSVEGKQNLVIMGRKTWFSIPEKNRPLKDRINIVLSRELKEPPRGAHFLAKSLDDALRLIEQPELASKVDMVWIVGGSSVYQEAMNQPGHLRLFVTRIMQEFESDTFFPEIDLGKYKLLPEYPGVLSEVQEEKGIKYKFEVYEKKD

SEQ ID NO: 34 - DNA, 7039 bp, Artificial Sequence
  Description of Artificial Sequence Synthetic polynucleotide
tcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacgggccctctagactcgagcggccgccactgtgctggagccgccaccatggactggacctggagggtgttctgcctgcttgcagtggcccccggagcccacagccaggttcagctgcagcagtctggacctgagctggtgaagcctggggctttagtgaagatatcctgcaaggcttctggttacaccttcacaaactacgatatacactgggtgaagcagaggcctggacagggacttgagtggattggatggatttatcctggagatggtagtactaagtacaatgagaaattcaagggcaaggccacactgactgcagacaaatcctccagcacagcctacatgcacctcagcagcctgacttctgagaaatctgcagtctatttctgtgcaagagagtgggcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgaaaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctcctggtagtagttcaatggctaagctgattgcgctcaccctcttggggatgggactggcactcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttacaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaacagaccgaacccgagctcggtaccaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcaggccgccaccatggagacccccgcccagctgctgttcctgttgctgctttggcttccagatactaccggcgacatccagatgacccagtctccatcctccttatctgcctctctgggagaaagagtcagtctcacttgtcgggcaagtcaggacattggtggtaacttatactggcttcagcagggaccagatggaactattaaacgcctgatctacgccacatccagtttagattctggtgtccccaaaaggttcagtggcagtaggtctgggtcagattattctctcaccatcagcagccttgagtctgaagattttgtagactattactgtctacagtattctagttctccgtggacgttcggtggaggcacaaagctggaaataaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagcgggaggtaccgagctcttacgcgtgctagctcgagatctgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttatcgattctagaagccgccaccatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatccttcgcgggacgtcctttgtttacgtcccgtcggcgctgaatcccgcggacgacccctcgcggggccgcttgggactctctcgtccccttctccgtctgccgttccagccgaccacggggcgcacctctctttacgcggtctccccgtctgtgccttctcatctgccggtccgtgtgcacttcgcttcacctctgcacgttgcatggagaccaccgtgaacgcccatcagatcctgcccaaggtcttacataagaggactcttggactcccagcaatgtcaacgaccgaccttgaggcctacttcaaagactgtgtgtttaaggactgggaggagctgggggaggagattaggttaaaggtctttgtattaggaggctgtaggcataaattggtctgcgcaccagcaccatgcaactttttcacctctgcctaatcatctcttgtacatgtcccactgttcaagcctccaagctgtgccttgggtggctttggggcatggacattgacccttataaagaatttggagctactgtggagttactctcgtttttgccttctgacttctttccttccgtcagagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcg

SEQ ID NO: 35 - protein, 819 aa, Artificial Sequence; identical to SEQ ID NO: 29
  Description of Artificial Sequence Synthetic polypeptide

SEQ ID NO: 36 - protein, 234 aa, Artificial Sequence; identical to SEQ ID NO: 27
  Description of Artificial Sequence Synthetic polypeptide

SEQ ID NO: 37 - protein, 187 aa, Artificial Sequence; identical to SEQ ID NO: 33
  Description of Artificial Sequence Synthetic polypeptide

SEQ ID NO: 38 - protein, 15 aa, Artificial Sequence
  Description of Artificial Sequence Synthetic peptide
GGGGSGGGGSGGGGS

SEQ ID NO: 39 - protein, 17 aa, Artificial Sequence
  Description of Artificial Sequence Synthetic peptide
SGGGGSGGGGSGGGGSS

SEQ ID NO: 40 - DNA, 25 bp, Artificial Sequence
  Description of Artificial Sequence Synthetic oligonucleotide
cactcttcaggaaccaccagtcttc

SEQ ID NO: 41 - protein, 804 aa, Artificial Sequence
  Description of Artificial Sequence Synthetic polypeptide
MDWTWRVFCLLAVAPGAHSQVQLQQSGPELVKPGALVKISCKASGYTFTNYDIHWVKQRPGQGLEWIGWIYPGDGSTKYNEKFKGKATLTADKSSSTAYMHLSSLTSEKSAVYFCAREWAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSSSLFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLQSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 42 - protein, 19 aa, Artificial Sequence
  Description of Artificial Sequence Synthetic peptide
MDWTWRVFCLLAVAPGAHS

SEQ ID NO: 43 - protein, 25 aa, Artificial Sequence
  Description of Artificial Sequence Synthetic peptide
QVQLQQSGPELVKPGALVKISCKAS

SEQ ID NO: 44 - protein, 10 aa, Artificial Sequence
  Description of Artificial Sequence Synthetic peptide
GYTFTNYDIH

SEQ ID NO: 45 - protein, 14 aa, Artificial Sequence
  Description of Artificial Sequence Synthetic peptide
WVKQRPGQGLEWIG

SEQ ID NO: 46 - protein, 17 aa, Artificial Sequence
  Description of Artificial Sequence Synthetic peptide
WIYPGDGSTKYNEKFKG

SEQ ID NO: 47 - protein, 32 aa, Artificial Sequence
  Description of Artificial Sequence Synthetic polypeptide
KATLTADKSSSTAYMHLSSLTSEKSAVYFCAR

SEQ ID NO: 48 - protein, 4 aa, Artificial Sequence
  Description of Artificial Sequence Synthetic peptide
EWAY

SEQ ID NO: 49 - protein, 11 aa, Artificial Sequence
  Description of Artificial Sequence Synthetic peptide
WGQGTLVTVSA

SEQ ID NO: 50 - protein, 98 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 51 - protein, 12 aa, Artificial Sequence
  Description of Artificial Sequence Synthetic peptide
EPKSCDKTHTCP

SEQ ID NO: 52 - protein, 113 aa, Artificial Sequence
  Description of Artificial Sequence Synthetic polypeptide
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 53 - protein, 107 aa, Artificial Sequence
  Description of Artificial Sequence Synthetic polypeptide
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 355 aa, Homo sapiens [sequence omitted]

SEQ ID NO: 55 - protein, 20 aa, Artificial Sequence
  Description of Artificial Sequence Synthetic peptide
METPAQLLFLLLLWLPDTTG

SEQ ID NO: 56 - protein, 23 aa, Artificial Sequence
  Description of Artificial Sequence Synthetic peptide
DIQMTQSPSSLSASLGERVSLTC

SEQ ID NO: 57 - protein, 11 aa, Artificial Sequence
  Description of Artificial Sequence Synthetic peptide
RASQDIGGNLY

SEQ ID NO: 58 - protein, 15 aa, Artificial Sequence
  Description of Artificial Sequence Synthetic peptide
WLQQGPDGTIKRLIY

SEQ ID NO: 59 - protein, 7 aa, Artificial Sequence
  Description of Artificial Sequence Synthetic peptide
ATSSLDS

SEQ ID NO: 60 - protein, 32 aa, Artificial Sequence
  Description of Artificial Sequence Synthetic polypeptide
GVPKRFSGSRSGSDYSLTISSLESEDFVDYYC

SEQ ID NO: 61 - protein, 9 aa, Artificial Sequence
  Description of Artificial Sequence Synthetic peptide
LQYSSSPWT

SEQ ID NO: 62 - protein, 11 aa, Artificial Sequence
  Description of Artificial Sequence Synthetic peptide
FGGGTKMEIKR

SEQ ID NO: 63 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC